(19) United States
(12) Patent Application Publication
(10) Pub. No.: US 2025/0242154 A1
(43) Pub. Date: Jul. 31, 2025

(54) Device and Method for Delivery of Transcutaneous Current

(71) Applicant: Exact Neuro LLC, Napa, CA (US)

(72) Inventor: Arkady Glukhovsky, Santa Clarita, CA (US)

(21) Appl. No.: 18/726,392

(22) PCT Filed: Jan. 2, 2023
(86) PCT No.: PCT/US2023/060005

§ 371 Date: Jul. 2, 2024

Related U.S. Application Data
(60) Provisional application No. 63/295,907, filed on Jan. 2, 2022.

(51) Int. Cl. A61N 1/05; A61N 1/36

(52) U.S. Cl. CPC A61N 1/0551; A61N 1/36057

(57) ABSTRACT

An implantable device may receive an alternating transcutaneous current, may rectify the alternating transcutaneous current to generate a positive current, may rectify the alternating transcutaneous current to generate a negative current, may stimulate tissue at a first location utilizing the positive current, and may stimulate tissue at a second location utilizing the negative current.

### CROSS REFERENCE TO RELATED APPLICATIONS

This patent application claims the benefit of and priority to U.S. Provisional Patent Application Ser. No. 63/295,907, entitled DEVICE AND METHOD FOR ELECTRICAL STIMULATION, filed Jan. 2, 2022, the contents of which are hereby incorporated by reference in their entirety. This application is related to co-pending International patent application Serial No. ______/______, entitled DEVICE AND METHOD FOR CURRENT STEERING IN AN IMPLANTABLE DEVICE BY AN EXTERNAL GENERATOR, filed contemporaneously herewith on Jan. 2, 2023, and co-pending International patent application Serial No. ______/______, entitled DEVICE AND METHOD FOR EVALUATING COUPLING WITH AN IMPLANTABLE DEVICE, filed contemporaneously herewith on Jan. 2, 2023, the contents of which are hereby incorporated by reference in their entirety.

### BACKGROUND

### Technical Field

The present disclosure relates generally to medical devices and treatments, and more particularly, to a partially implantable system for delivery of transcutaneous current.

### Introduction

Implantable devices can be implanted into a tissue of a subject, such as a human patient, to apply electrical current to nerves or tissue and to record or sense neural signals emanating from the central and peripheral nervous system. Implantable devices can be used for a variety of clinical indications, including, without limitation, electrical stimulation and/or sensing for treatment of pain, incontinence, motor response, treatment of headache and migraine.

Some systems utilize an external device to deliver energy transcutaneously to an implanted device. In some systems, the external device may apply low frequency electrical current to deliver power and/or energy to an implanted device, similar to the transcutaneous energy transfer and application used in devices for delivering transcutaneous electrical stimulation (also known as TENS devices). When electrical current is delivered to the body of a subject at a low frequency, the electrical current can cause activation of the nerves and/or tissue causing pain, muscle contraction, discomfort, and other undesirable sensations or responses.

In some systems, the external device may apply high frequency transcutaneous current to deliver power and/or energy to an implanted device. Some systems utilize high frequency transcutaneous current or bursts of high frequency current to deliver energy to the implant. The implant rectifies the delivered high frequency current, and delivers the rectified current to the targeted nerve. The disadvantage of this approach is that rectification allows only the positive or only the negative fraction of the current to be delivered to the targeted nerve or tissue, while the other fraction or the current is wasted.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

In an aspect of the disclosure, an apparatus is provided. The apparatus may be an implantable device. The apparatus may include a receiving electrode, a first output electrode, a first channel, a second output electrode, and a second channel. The implantable device may be configured to receive a transcutaneous current having a first portion and a second portion. The first channel may be configured to allow the first portion of the transcutaneous current to pass from the receiving electrode to the first output electrode and to inhibit the second portion of the transcutaneous current passing from the first output electrode to the receiving electrode, and the second channel may be configured to allow the second portion of the transcutaneous current to pass from the second output electrode to the receiving electrode and to inhibit the first portion of the transcutaneous current passing from the receiving electrode to the second output electrode.

In some aspects, the transcutaneous current is an alternating transcutaneous current, the first portion of the transcutaneous current is a positive portion of the alternating transcutaneous current, and the second portion of the transcutaneous current is a negative portion of the alternating transcutaneous current.

In some aspects, the transcutaneous current comprises a series of bursts of alternating transcutaneous current, the first portion of the transcutaneous current is a positive portion of the series of bursts of alternating transcutaneous current, and the second portion of the transcutaneous current is a negative portion of the series of bursts of alternating transcutaneous current.

In some aspects, the implantable device is configured to receive the transcutaneous current from an external generator.

In some aspects, the transcutaneous current received by the implantable device is a fraction of a current applied by the external generator.

In some aspects, the implantable device is configured to receive transcutaneous current from an external generator through electrically conductive coupling.

In some aspects, the electrically conductive coupling is electrically coupling through conductive body tissue.

In some aspects, the implantable device is configured to: receive a burst of alternating transcutaneous current from an external generator, the burst of alternating transcutaneous current burst having a positive portion and a negative portion; rectify the burst of alternating transcutaneous current to generate a positive current burst; rectify the burst of alternating transcutaneous current to generate a negative current burst; stimulate tissue at an implantation site of the first output electrode utilizing the positive current burst; and stimulate tissue at an implantation site of the second output electrode using the negative current burst.

In some aspects, the implantable device is configured to: receive a high frequency transcutaneous current from an external generator, the high frequency transcutaneous current burst having a positive portion and a negative portion; rectify the high frequency transcutaneous current to generate a positive high frequency current; rectify the high frequency transcutaneous current to generate a negative high frequency current; stimulate tissue at an implantation site of the first output electrode utilizing the positive high frequency current; and stimulate tissue at an implantation site of the second output electrode using the negative high frequency current.

In some aspects, the first channel comprises a first rectification element configured to inhibit current from flowing from the first output electrode to the receiving electrode.

In some aspects, the second channel comprises a second rectification element configured to inhibit current from flowing from the receiving electrode to the second output electrode.

In some aspects, the rectification element is a diode.

In some aspects, the first channel comprises a capacitor coupled between the first output electrode and the receiving electrode, a diode coupled between the first output electrode and the receiving electrode, and a resistor coupled in parallel to the diode.

In some aspects, the second channel comprises a second capacitor coupled between the second output electrode and the receiving electrode, a second diode coupled between the second output electrode and the receiving electrode, and a second resistor coupled in parallel to the second diode.

In some aspects, the first output electrode and the second output electrode are configured to be implanted in proximity to a nerve of a subject, and the receiving electrode is configured to be implanted in proximity to a surface of the skin of the subject.

In some aspects, the implantable device is configured to apply monopolar stimulation to the nerve at an implantation site of the first output electrode, is configured to apply monopolar stimulation to the nerve at an implantation site of the second output electrode, and is configured to apply quasi-bipolar stimulation to the nerve between the implantation site of the first output electrode and the implantation site of the second output electrode based on applying monopolar stimulation to the tissue at the first location and applying monopolar stimulation at the second location.

In some aspects, the implant includes a bypass electrode, and a bypass channel configured to allow transcutaneous current to pass between the receiving electrode and the bypass electrode, and the bypass channel is configured to allow current to pass from the receiving electrode to the bypass electrode and to pass from the bypass electrode to the receiving electrode.

In some aspects, the first output electrode and the second output electrode are between the receiving electrode and the bypass electrode, and the implantable device is configured to stimulate an efferent nerve.

In some aspects, the bypass electrode is between the receiving electrode and the first output electrode, and the implantable device is configured to stimulate an afferent nerve. In some aspects, the bypass electrode is between the receiving electrode and the first output electrode, and the implantable device is configured to stimulate a nerve containing both efferent and afferent fibers. In some aspects, the bypass channel is configured to deliver alternating transcutaneous current to inhibit activation of the nerve.

In some aspects, the bypass channel is configured to deliver low frequency current for nerve stimulation, and the first channel is configured to deliver rectified alternating current for nerve stimulation.

In some aspects, the bypass channel comprises a tuning resistor configured to determine the ratio of current applied by the bypass channel relative to the first channel.

In some aspects, the implantable device includes an implantable lead, the implantable lead comprises a first end and a second end opposite the first end, the receiving electrode is located at or near the first end of the implantable lead, and the first output electrode or the second output electrode is located at or near the second end of the implantable lead.

In some aspects, the first output electrode and the second output electrode are located at or near the second end of the implantable lead.

In some aspects, the implantable device includes an implantable lead, wherein the implantable lead comprises a first end, a first branch with a second end, and a second branch with a third end, the receiving electrode is located at or near the first end of the implantable lead, the first output electrode is located at or near the second end of the implantable lead, and the second output electrode is located at or near the third end of the implantable lead.

In some aspects, the implantable device includes a receiver, wherein the receiver comprises a cavity, the first channel comprises a rectification element configured to rectify current flowing in the first channel, and the rectification element is in the cavity of the receiver. In some aspects, the second channel comprises a second rectification element configured to rectify current flowing in the second channel, the second rectification element being in the cavity of the receiver.

In some aspects, the receiver further comprises a lead connector.

In some aspects, the implantable device includes a receiver, the receiver comprising the receiving electrode and a cavity, and electrical elements configured to rectify the electrical current flowing in the first or in the second channel, the electrical elements being in the cavity of the receiver.

In some aspects, the receiver comprises an electrical and mechanical connector configured to attach and electrically connect to a first end of an implanted lead.

In some aspects, the first end of the lead electrically connects the first and the second channels to the second end of the lead.

In some aspects, the transcutaneous current is a burst of alternating transcutaneous current with a carrier frequency between 5 kHz and 100 kHz.

In some aspects, the transcutaneous current is a burst of alternating transcutaneous current with a carrier frequency between 100 kHz and 5 MHz.

In some aspects, the transcutaneous current is a burst of alternating transcutaneous current with a carrier frequency between 1 kHz and 5 kHz.

In some aspects, the transcutaneous current is a burst of alternating transcutaneous current and the carrier frequency of the alternating transcutaneous current has a first value at a first time during the burst of alternating transcutaneous current and has a second value at a second time during the burst of alternating transcutaneous current, the first value being different than the second value.

In some aspects, the transcutaneous current comprises a first burst of alternating transcutaneous current and a second burst of alternating transcutaneous current, and a carrier frequency of the transcutaneous current during the first burst is different than a carrier frequency of the transcutaneous current during the second burst.

In another aspect of the disclosure, a method of applying current to a tissue of a subject is provided. The method may be performed by an implantable device. The method may include receiving, by an implantable device, an alternating transcutaneous current; rectifying, by the implantable device, the alternating transcutaneous current to generate a positive current; rectifying, by the implantable device, the alternating transcutaneous current to generate a negative current; stimulating, by the implantable device, tissue at a first location utilizing the positive current; and stimulating, by the implantable device, tissue at a second location utilizing the negative current.

In some aspects, the alternating transcutaneous current comprises a positive portion and a negative portion, the implantable device comprises a first channel configured to allow the positive portion of the alternating transcutaneous current to pass from a receiving electrode of the implantable device to a first output electrode of the implantable device and to inhibit the negative portion of the alternating transcutaneous current passing from the first output electrode to the receiving electrode, the first output electrode being at the first location, the implantable device comprises a second channel configured to allow the negative portion of the transcutaneous current to pass from a second output electrode of the implantable device to the receiving electrode and to inhibit the positive portion of the transcutaneous current passing from the receiving electrode to the second output electrode.

In some aspects, the first output electrode and the second output electrode are configured to be implanted in proximity to a nerve of a subject, and the receiving electrode is configured to be implanted in proximity to a surface of the skin of the subject.

In some aspects, the method may include applying, by the implantable device, an unrectified current to tissue at a third location.

In some aspects, the implantable device comprises a bypass channel configured to allow transcutaneous current to pass between a receiving electrode of the implantable device and a bypass electrode of the implantable device and to pass from the bypass electrode to the receiving electrode.

In some aspects, the first output electrode and the second output electrode are between the receiving electrode and the bypass electrode, and the implantable device is configured to stimulate an efferent nerve.

In some aspects, the bypass electrode is between the receiving electrode and the first output electrode, and the implantable device is configured to stimulate an afferent nerve.

In some aspects, the bypass electrode is between the receiving electrode and the first output electrode, and the implantable device is configured to stimulate a nerve containing both efferent and afferent fibers.

In some aspects, the bypass channel is configured to deliver alternating transcutaneous current to inhibit activation of the nerve.

In some aspects, the bypass channel is configured to deliver low frequency current for nerve stimulation, and the first channel is configured to deliver rectified alternating current for nerve stimulation.

In some aspects, the bypass channel comprises a tuning resistor configured to determine the ratio of current applied by the bypass channel relative to the first channel.

In some aspects, the alternating transcutaneous current comprises a series of bursts of alternating transcutaneous current, the positive portion of the alternating transcutaneous current is a positive portion of the series of bursts of alternating transcutaneous current, and the negative portion of the transcutaneous current is a negative portion of the series of bursts of alternating transcutaneous current.

In some aspects, the alternating transcutaneous current is received from an external generator.

In some aspects, the alternating transcutaneous current received by the implantable device is a fraction of a current applied by the external generator.

In some aspects, the alternating transcutaneous current is received through electrically conductive coupling.

In some aspects, the electrically conductive coupling is electrically coupling through conductive body tissue.

In some aspects, the alternating transcutaneous current is high frequency alternating transcutaneous current, the positive current is a positive high frequency current, and the negative current is a negative high frequency current.

In some aspects, stimulating tissue at the first location comprises applying monopolar stimulation to the tissue at the first location, stimulating tissue at the second location comprises applying monopolar stimulation to the tissue at the second location, and the method includes applying quasi-bipolar stimulation between the first location and the second location based on applying monopolar stimulation to the tissue at the first location and applying monopolar stimulation at the second location.

In some aspects, the alternating transcutaneous current is a burst of alternating transcutaneous current with a carrier frequency between 5 kHz and 100 kHz.

In some aspects, the alternating transcutaneous current is a burst of alternating transcutaneous current with a carrier frequency between 100 kHz and 5 MHz.

In some aspects, the alternating transcutaneous current is a burst of alternating transcutaneous current with a carrier frequency between 1 kHz and 5 kHz.

In some aspects, the alternating transcutaneous current is a burst of alternating transcutaneous current and the carrier frequency of the alternating transcutaneous current has a first value at a first time during the burst of alternating transcutaneous current and has a second value at a second time during the burst of alternating transcutaneous current, the first value being different than the second value.

In some aspects, the transcutaneous current comprises a first burst of alternating transcutaneous current and a second burst of alternating transcutaneous current, and a carrier frequency of the transcutaneous current during the first burst is different than a carrier frequency of the transcutaneous current during the second burst.

In some aspects, the transcutaneous current comprises a first burst of alternating transcutaneous current and a second burst of alternating transcutaneous current, and a carrier waveform of the transcutaneous current during the first burst is different than a carrier waveform of the transcutaneous current during the second burst.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating a low frequency waveform applied by an external generator to transcutaneously deliver current through an implanted device.

FIG. 2A is a diagram illustrating a system for applying low frequency transcutaneous electrical stimulation.

FIG. 2B is a diagram illustrating a low frequency current pulse applied by the external generator.

FIG. 2C is a diagram illustrating current passing through the implantable device resultant from the low frequency current pulse.

FIG. 3 is a diagram illustrating a high frequency stimulation waveform applied by an external generator to deliver current through an implanted device.

FIG. 4A is a diagram illustrating a system for applying high frequency transcutaneous electrical energy in the form of transcutaneous electrical current.

FIG. 4B is a diagram illustrating a burst of high frequency current applied by the external generator.

FIG. 4C is a diagram illustrating current passing through the implantable device resultant from the high frequency current burst.

FIG. 5 is a diagram illustrating an embodiment of a rectification circuit for the implantable device described with respect to FIG. 4A.

FIG. 6A is a circuit diagram illustrating current flow in the implantable device of FIG. during the burst phase of transcutaneous stimulation.

FIG. 6B is a circuit diagram illustrating current flow in the implantable device of FIG. 5 during the discharge phase of transcutaneous stimulation.

FIG. 7 is a diagram illustrating a multi-channel system for applying transcutaneous current.

FIG. 8 is a circuit diagram of a multi-channel implantable device for applying transcutaneous current.

FIG. 9A is a circuit diagram illustrating current flow in the second channel of the implantable device of FIG. 8 during the burst phase of transcutaneous stimulation.

FIG. 9B is a circuit diagram illustrating current flow in the second channel of the implantable device of FIG. 8 during the discharge phase of transcutaneous stimulation.

FIG. 10 a diagram illustrating stimulation of a nerve utilizing monopolar and quasi-bipolar stimulation.

FIG. 11 is a circuit diagram of an implantable device including two channels and a bypass channel.

FIG. 12 is a circuit diagram of an implantable device including two channels and a bypass channel.

FIG. 13 is a diagram illustrating a system for applying multi-channel transcutaneous current utilizing a branched lead.

FIG. 14A is a diagram illustrating the implantable device with the receiver and the lead in a separated state.

FIG. 14B is a diagram illustrating the implantable device with the lead coupled to the receiver.

FIG. 15A is a diagram illustrating the implantable device with the receiver and the branched lead in a separated state.

FIG. 15B is a diagram illustrating the implantable device with the branched lead coupled to the receiver.

FIG. 16 is a diagram illustrating an implantable device utilizing an adapter.

FIG. 17 is a diagram illustrating an implantable device utilizing a branched adapter.

FIG. 18 is a flowchart illustrating a method of applying current to a tissue of a subject.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components may be shown in block diagram form in order to avoid obscuring such concepts.

Several aspects of medical devices will now be presented with reference to various apparatus and methods.

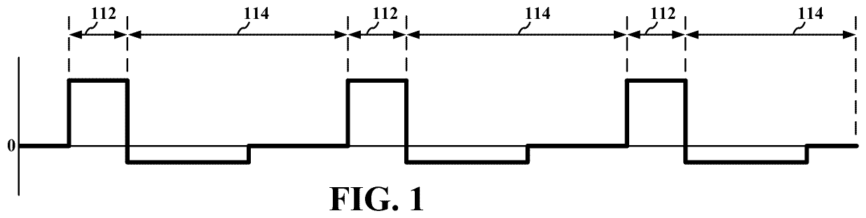

FIG. 1 is a diagram illustrating a low frequency waveform applied by an external generator to transcutaneously deliver current through an implanted device. The external generator may apply current having the waveform to the skin of a subject at the implantation site of the implanted device. The waveform includes burst phases 112 and discharge phases 114. The external generator applies a low frequency pulse of current during each burst phase 112. For example, the external generator may deliver a single pulse of current for the duration of each burst phase 112. A pulse may be a simple rectangular pulse, as shown in FIG. 1. A pulse may alternatively have another shape, such as a single sawtooth pulse, a single triangular pulse, or a single half sine pulse, or any other arbitrary waveform. In some aspects, the shape can change from pulse to pulse.

During the discharge phases 114, the external generator may not deliver current, which may allow for capacitors in the implanted device to discharge and for charge in stimulated nerves and tissue to dissipate. The external generator may deliver voltage/current with opposite polarity, lower amplitude, and over a longer duration than the pulse of current during some or all of the discharge phase 114, as shown in FIG. 1, so that the voltage/current waveform may be charge balanced. Note that the discharge phase can have lower current amplitude or the same amplitude, or higher amplitude than the burst phase. Moreover, the burst phase may be not applied at all, resulting in charge unbalanced stimulation. The low frequency delivery of current pulses may be repeated with a frequency of 1 Hz to hundreds of Hz.

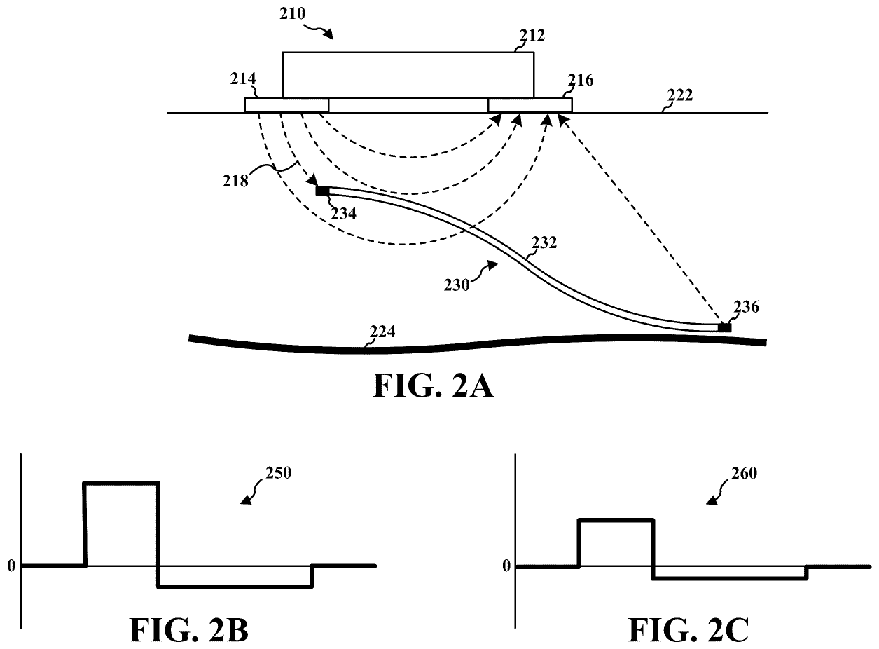

FIG. 2A is a diagram illustrating a system for applying low frequency transcutaneous electrical stimulation. The system includes an external generator 210 and an implantable device 230. The external generator 210 includes a device housing 212, a source electrode 214, and a return electrode 216. The source electrode 214 and the return electrode 216 may include hydrogel, wetted cloth, metal, conductive polymers, or other electrically conductive materials. The implantable device 230 includes a receiving electrode 234, an output electrode 236, and a lead 232 between the receiving electrode 234 and the output electrode 236. The receiving electrode 234 and the output electrode 236 are exposed, allowing them to contact the tissue at their respective implantation sites. The receiving electrode 234 is electrically connected to the output electrode 236 through a conductor inside the lead 232.

The implantable device 230 may be implanted beneath the skin 222 of a subject. The output electrode 236 may be implanted in close proximity to a nerve 224. The receiving electrode 234 may be implanted closer to the surface of the skin 222.

The external generator 210 may be placed on the skin of a subject, with the source electrode 214 and the return electrode 216 in contact with the subject's skin and positioned over the implantation site of the receiving electrode 234. The external generator 210 may deliver low frequency current pulses between the source electrode 214 and the return electrode 216. For example, the external generator 210 may deliver low frequency current pulses as described above with respect to FIG. 1. As a result of the delivered current pulses, current may flow between the source electrode 214 and the return electrode 216 through the skin 222 and the underlying tissue.

The implant 230 presents a conductive path between the source electrode 214 and the return electrode 216 for some of the current applied to the skin by the external generator 210. Some of the current applied by the external generator 210 passes between the source electrode 214 and the return electrode 216 through the tissue of the subject without passing through the implantable device 230. A portion 218 of the current passes from the source electrode 214 to the receiving electrode 234 through the tissue of the subject, through the conductor in the lead 232 to the output electrode 236, and from the output electrode 236 to the return electrode 216 through the tissue of the subject. The current passing from the output electrode 236 to the return electrode 216 may stimulate the nerve 224 due to the proximity of the output electrode 236 to the nerve 224.

FIG. 2B is a diagram illustrating a low frequency current pulse 250 applied by the external generator 210. FIG. 2C is a diagram illustrating current 260 passing through the implantable device 230 resultant from the low frequency current pulse 250. The current 260 may have the same shape as or a similar shape to the low frequency current pulse 250 but may have a lower amplitude as only a fraction of the current applied by the external stimulator 210 passes through the implantable device 230. The ratio of the peak of the current passing through an implantable device (e.g., the peak of current 260) to the peak of the current applied by an external stimulator (e.g., the peak of the low frequency current pulse 250) is referred to herein as the pick-up ratio. The pick-up ratio for a particular external generator and implantable device may vary based on factors including the shape and size of the receiving electrode, the depth of implantation of the receiving electrode, and the alignment between the external generator electrodes and the receiving electrode.

When an external generator delivers current at or above a first level, the system may achieve a desired therapeutic effect through stimulation at the target nerve or tissue. When an external generator delivers current at or above a second level, the subject may experience an undesired effect such as pain, an unpleasant feeling, or undesired contractions in the tissue underlying the electrodes of the external generator. Therapeutic window is defined herein as the difference in amplitude between the level of current (or voltage) which causes the desired therapeutic effect and the level of stimulating current (or voltage) that causes the undesired effect.

The system for applying low frequency transcutaneous electrical stimulation depicted in FIG. 2A may have a relatively small therapeutic window, or may even have no therapeutic window (e.g., the system cannot achieve the therapeutic effect without causing the undesired effect. The current 260 at the output electrode 236 may need to be 1 mA to 3 mA to achieve the therapeutic effect, the pick up ratio may be between 10 and 20 percent, and the low frequency current pulse 250 when applied with an amplitude of 5 mA to 30 mA may cause an undesired effect.

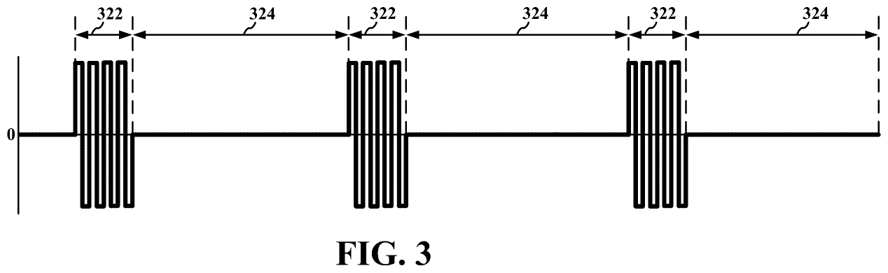

FIG. 3 is a diagram illustrating a high frequency waveform applied by an external generator to deliver current through an implanted device. The waveform includes burst phases 322 and discharge phases 324. The external generator applies a burst of high frequency alternating current between its electrodes during each burst phase 322. Applying a burst of high frequency alternating current may refer to applying current which alternates between positive and negative at a high frequency. In some aspects, the external generator may apply a burst comprised of high frequency rectangular pulses, as shown in FIG. 3. The burst of high frequency current pulse may alternatively have another shape, such as a high frequency sinusoidal wave, a high frequency sawtooth wave, a high frequency triangle wave, or high frequency wave of arbitrary shape. High frequency may refer to a waveform having a frequency between tens of kHz to above several MHz. In some aspects, high frequency may refer to the alternating current in the burst of alternating current having a frequency between 1 kHz and 5 kHz. In some aspects, high frequency may refer to the alternating current in the burst of alternating current having a frequency between 5 kHz and 100 kHz. In some aspects, high frequency may refer to the alternating current in the burst of alternating current having a frequency between 100 kHz and 5 MHz. In some aspects, the alternating current in the burst of alternating current may have a frequency that varies over the duration of the burst phase. In some aspects, the alternating current in one burst of the transcutaneous current may have a different frequency than the alternating current in another burst of the transcutaneous current. In some aspects, the alternating current in the burst of alternating current may have a carrier waveform which changes during the duration of the burst. In some aspects, the alternating current in one burst of the transcutaneous current may have a different carrier waveform than the alternating current in another burst of the transcutaneous current.

During the discharge phases 324, the external generator may apply no current. The burst of high frequency current may be charged balanced, so it may be unnecessary for the external generator to apply charge balancing during the discharge phases 324. The bursts of high frequency current may be repeated with a frequency of 1 Hz to hundreds of Hz.

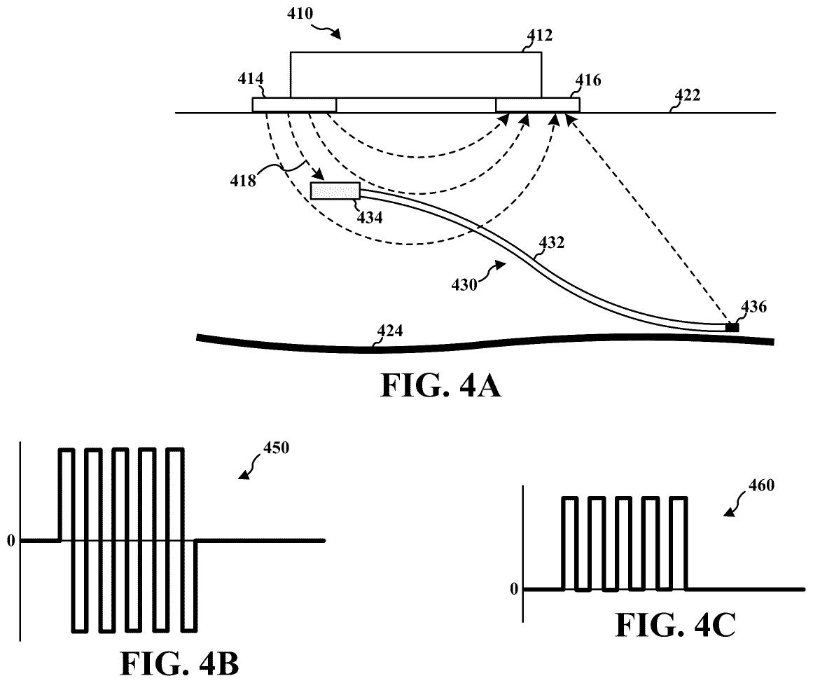

FIG. 4A is a diagram illustrating a system for applying high frequency transcutaneous electrical energy in the form of transcutaneous electrical current. The system includes an external generator 410 and an implantable device 430. The external generator 410 includes a device housing 412, a source electrode 414, and a return electrode 416. The implantable device 430 includes a receiver 434, an output electrode 436, and a lead 432 between the receiver 434 and the output electrode 436. The receiver 434 may be a hermetically sealed enclosure. The receiver 434 may include a receiving electrode exposed to the surrounding tissue. For example, the receiver housing may include a conductive material which may function as a receiving electrode. The receiving electrode and the output electrode 436 are exposed, allowing them to contact the tissue at their respective implantation sites. The receiver 434 may include an electrical circuit. The receiving electrode is electrically connected to the output electrode 436 through the electrical circuit and an insulated conductor inside the lead 432.

The external generator 410 may apply bursts of high frequency current between the source electrode 414 and the return electrode 416. For example, the external generator 410 may deliver bursts of high frequency current as described above with respect to FIG. 3. A portion 418 of the current applied by the external generator 410 passes through the implantable device 430.

The implantable device 430 may include a rectification circuit. For example, the rectification circuit may be in the receiver 434. FIG. 4B is a diagram illustrating a burst of high frequency current 450 applied by the external generator 410. FIG. 4C is a diagram illustrating the current 460 passing through the implantable device 430 resultant from the high frequency current burst 450. The rectification circuit in the implantable device 430 allows current to pass from the receiving electrode at the receiver 434 to the output electrode 436 but inhibits or entirely prevents current flow from the output electrode 436 to the receiving electrode. Accordingly, the current 460 passing through the implantable device 430 may include the positive portions of the burst of high frequency current 450, but may not include the negative portions of the burst of high frequency current 450. Positive current (e.g., current passing from the output electrode to the return electrode) which stimulates a nerve or tissue surrounding the output electrode may be referred to hereinafter as anodic stimulation. The current 460 may also have a lower amplitude than the corresponding portions of the high frequency current pulse 450 as only a fraction of the current delivered by the external generator 410 passes through the implantable device 430 during the positive portions of the burst of high frequency current 450.

Alternatively, the rectification circuit in the implantable device 430 may be oriented to allow current to pass from the output electrode 436 to the receiving electrode but to inhibit or entirely prevent current flowing from the receiving electrode to the output electrode 436. In that configuration, the current passing through the implantable device 430 may include the negative portions of the burst of high frequency current 450, but may not include the negative portions of the burst of high frequency current 450. Negative current (e.g., current passing from the return electrode to the output electrode) which stimulates a nerve or tissue surrounding the output electrode may be referred to hereinafter as cathodic stimulation.

The sensitivity of tissues, including nerves, to stimulation may decrease as the carrier frequency of the pulses of current in the high frequency bursts of current increases. The tissue activation or excitation threshold increases as the carrier frequency of the pulses of current in the high frequency bursts increases, and higher amplitude current is required to activate the tissue. High frequency pulses may be applied to a subject without causing sensation or contractions, while low frequency pulses (e.g., those described above with respect to FIGS. 1-2) having the same intensity might cause sensation or contractions. The intensity of the current may refer to the amplitude or the duration of the pulse or the burst. Alternatively, the intensity of the current may refer to the electrical charge delivered during the pulse or the burst. Once rectified, only positive or only negative bursts are delivered to the nerve 424. Due to the high frequency of the delivered rectified bursts, the electrical charge delivered to the nerve does not have time to dissipate and accumulates at the nerve during the duration of the stimulation burst, resulting in nerve activation similar to that achieved by delivering low frequency stimulation (e.g., continuous stimulation for the duration of the pulse). The sensitivity (e.g., the activation threshold) of the nerves to the non-rectified stimulation is inversely proportional to frequency of the individual pulses in the burst. Once rectified, the electrical charge delivered by the current may be the same, irrespective of the frequency of the individual pulses in the burst. Thus the difference between the rectified and the non-rectified stimulation threshold increases with the frequency of the individual pulses in the burst. Accordingly, the system for applying high frequency transcutaneous current described with respect to FIGS. 4A-C may have a larger therapeutic window than the system for applying low frequency transcutaneous stimulation described with respect to FIGS. 2A-C.

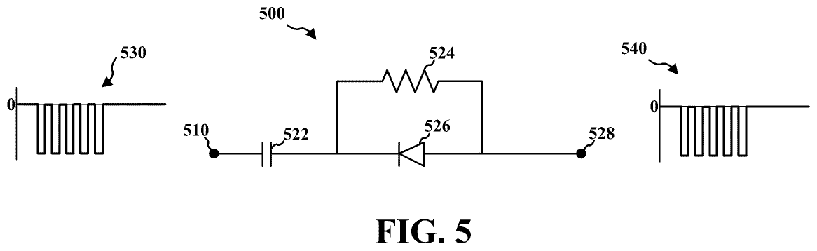

FIG. 5 is a diagram illustrating an embodiment of a rectification circuit 500 for the implantable device 530 described with respect to FIG. 4A. The rectification circuit 500 is connected between the receiving electrode 510 and the output electrode 528. The rectification circuit 500 includes a capacitor 522, a resistor 524, and a diode 526.

The rectification circuit 500 is configured for cathodic stimulation. The diode 526 is oriented to allow current to pass from the output electrode 528 to the receiving electrode 510. The diode 526 may be a Schottky diode.

The capacitor 522 is connected between the receiving electrode 510 and the diode 526, serving as a DC block to ensure that the stimulation delivered to the nerve through the implantable device is charge balanced. The capacitor 522 may have a capacitance suitable to allow for the high frequency rectified current pulse to pass through with minimal attenuation or distortion, while allowing for the capacitor to adequately or fully discharge during the discharge periods between stimulation periods. For example, the capacitance of the capacitor 522 may be on the order of several micro-Farads.

The resistor 524 is connected in parallel with the diode 526. The resistor 524 may provide a path for the capacitor 522 to discharge during the discharge periods. The resistor 524 may have a resistance suitable to present high impedance relative to the diode 526, such that little or no current flows through the resistor 524 during the stimulation periods, but to allow for the capacitor 522 to adequately discharge during the discharge periods. For example, the resistance of the resistor 524 may be on the order of several kilo-Ohms.

The rectification circuit 500 may include additional elements. For example, the rectification circuit 500 may include Electro-Static Discharge (ESD) protecting elements to protect the diode 526 from ESD damage, for example when the implant is being handled during the implantation procedure. As another example, the rectification circuit 500 may include components or circuits to improve the Magnetic Resonance (MR) compatibility of the implant. In some aspects, the rectification circuit 500 may include fewer elements. For example, the rectification circuit 500 may include only the diode 526. In some aspects, instead of a diode 526 the rectification circuit 500 may include another non-linear element, such as transistor.

When a burst of alternating transcutaneous current is applied to the implantable device 500, for example as described above with respect to FIG. 3, the diode 526 allows negative current to pass through the implantable device 500 but prevents some or all positive current passing through the implantable device 500. The current passing through the receiving electrode 510 is shown at 530 and the current passing through the output electrode 528 is shown at 540. As little or no positive current passes through the implantable device 500, the current at the receiving electrode 510 and the current at the output electrode 526 may be the same or may be similar. The continuous pulses of negative current at the receiving electrode 510 may accumulate a negative charge in the tissue surrounding the receiving electrode 510, and may cause unwanted activation or sensation in the tissue.

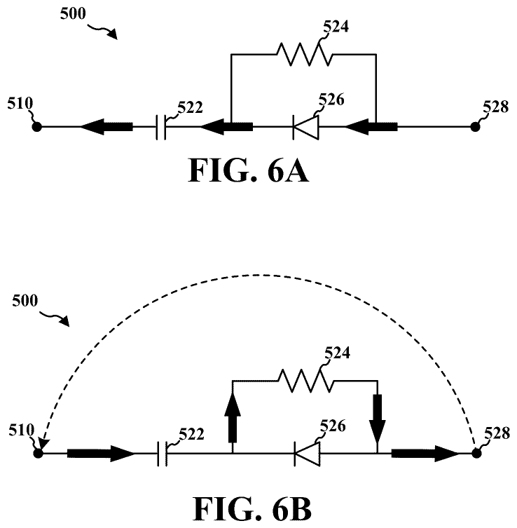

FIG. 6A is a circuit diagram illustrating current flow in the implantable device 500 of FIG. 5 during the burst phase of transcutaneous stimulation. Negative portions of the alternating transcutaneous current pass from the output electrode 528, through the diode 526 and through the capacitor 522, to the receiving electrode 510. The negative current passing through the capacitor 522 may build a charge on the capacitor over the duration of the burst. The impedance of the capacitor 524 may be high enough relative to tissue paths between the electrodes of the external generator that little or no positive current passes through the implantable device 500 during positive portions of the alternating transcutaneous current. FIG. 6B is a circuit diagram illustrating current flow in the implantable device 500 of FIG. 5 between the bursts (e.g., during the discharge phase) of transcutaneous current. The implantable device 500 is not exposed to transcutaneous current during this period. The charge accumulated on the capacitor 522 during the burst phase may discharge via current flowing from one terminal of the capacitor 522 through the resistor 524 to the output electrode 528, from the output electrode 528 to the receiving electrode 510 through tissue, and from the receiving electrode 510 to the other terminal of the capacitor 522.

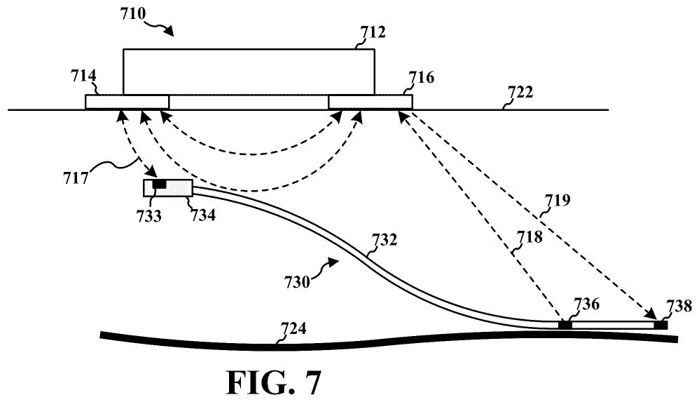

FIG. 7 is a diagram illustrating a multi-channel system for applying transcutaneous current. The system includes an external generator 710 and an implantable device 730. The external generator 710 includes a device housing 712, and two surface electrodes: a source electrode 714 and a return electrode 716. The external generator 710 may apply current between the source electrode 714 and the return electrode 716. The source electrode 714 and the return electrode 716 may include hydrogel, wetted cloth, metal, electrically conductive polymer, or other electrically conductive materials. The source electrode 714 and the return electrode 716 may be attached directly to the device housing 712, or one or both may be electrically connected to the device housing 712 and the external generator 710 by a wire (e.g., a lead).

The source electrode 714 and the return electrode 716 may be similar or identical electrodes attached to opposite ends of the current generating circuit; the names “source” and “return” are used herein for clarity to differentiate where the electrode is placed with respect to the implantable device, and to clarify the meaning of “positive” and “negative” current in a system for applying transcutaneous current. The source electrode 714 may be the electrode placed above the implantation site of a receiving electrode of the implantable device, and the return electrode 716 may be the electrode placed at a different location (e.g., above or near the implantation site of the output electrodes of the implantable device). Positive current, as used herein, refers to current passing from the source electrode (e.g., the electrode proximate the receiving electrode) to the return electrode (e.g., the electrode placed at a different location), and negative current, as used herein, refers to current passing from the return electrode to the source electrode. In some aspects, the electrodes of the external generator 710 may be interchangeable and whichever electrode is placed above the receiving electrode of the implantable device may be referred to as the source electrode as used herein.

The implantable device 730 includes a receiver 734, a receiving electrode 733, a first output electrode 736, a second output electrode 738, and a lead 732. In some aspects, the receiving electrode 733 (e.g., with the receiver 734) may be at or near one end of the lead 732, and the first output electrode 736 and the second output electrode 738 may be at the other end of the lead 732. The receiving electrode 733, the first output electrode 736, and the second output electrode 738 are exposed, allowing them to contact the tissue at their respective implantation sites, and may be connected by insulated conductors running inside the implantable device 730.

In some aspects, the first output electrode 736 and the second output electrode 738 may be stimulating electrodes. They may be implanted at locations targeted for delivery of stimulation by the system for applying transcutaneous current. The current passing through the first output electrode 736 and the second output electrode 738 may stimulate the tissue surrounding the electrodes (e.g., may activate a nerve at the implantation site of the electrode).

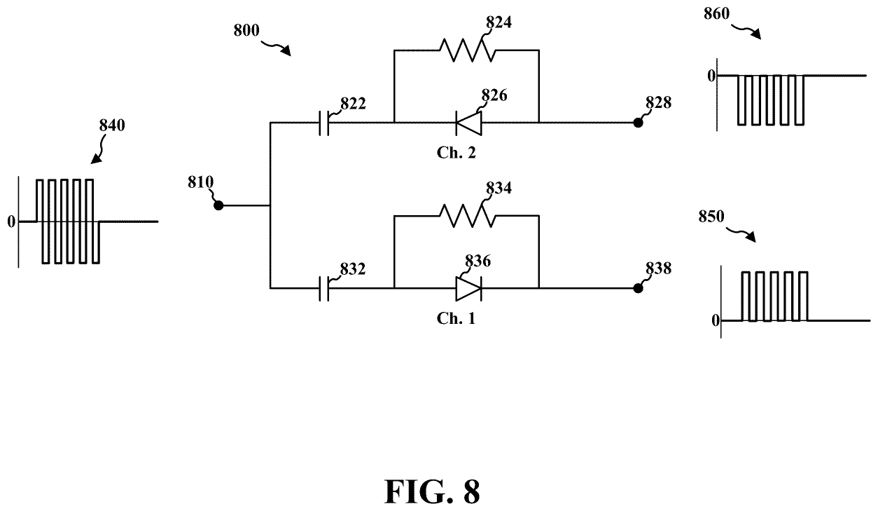

The implantable device 730 includes a first channel and a second channel. The first and second channels may be separate electrical paths for delivering stimulation current through the implantable device. The first channel connects the receiving electrode 733 to the first output electrode 736 via the electronic circuit such as shown in FIG. 8 and may allow current to pass between the receiving electrode 733 and the first output electrode 736. The second channel connects the receiving electrode 733 to the second output electrode 738 via the electronic circuit such as shown in FIG. 8 and may allow current to pass between the receiving electrode 733 and the second output electrode 738.

The implantable device 730 may include a first rectification element and a second rectification element. The first channel may include the first rectification element and the second channel may include the second rectification element. The first rectification element may be electrically connected to the receiving electrode 733 and the first stimulation electrode 736, and may be configured to allow current to pass from the receiving electrode 733 to the first output electrode 736, and to inhibit current from flowing from the first output electrode 736 to the receiving electrode 733 (e.g., may be configured for anodic stimulation). The second rectification element may be electrically connected to the receiving electrode 733 and the second stimulation electrode 738, and may be configured to allow current to pass from the second stimulation electrode 738 to the receiving electrode 733, and to inhibit current from flowing from the receiving electrode 733 to the second stimulation electrode 738 (e.g., may be configured for cathodic stimulation).

The receiving electrode 733 may be coupled to the receiver 734. For example, some or all of the housing of the receiver 734 may include a conductive material which may function as the receiving electrode 733. The receiver 734 may include a hermetically sealed enclosure, and the first rectification element and the second rectification element may be contained within the receiver 734.

The receiving electrode 733 is electrically connected to the first output electrode 736 through a first rectification circuit and an insulated conductor inside the lead 732. The receiving electrode 733 is electrically connected to the second output electrode 738 through a second rectification circuit and a second insulated conductor inside the lead 732.

The implantable device 730 may be implanted beneath the skin 722 of a subject. The first output electrode 736 and the second output electrode 738 may be implanted in close proximity to a nerve 724. The receiving electrode 733 may be implanted closer to the surface of the skin 722.

The external generator 710 may be placed on the skin 722 of a subject, with the source electrode 714 and the return electrode 716 in contact with the subject's skin 722 and with the source electrode 714 positioned over the implantation site of the receiving electrode 733. The external stimulator 710 may apply bursts of high frequency current between the source electrode 714 and the return electrode 716. For example, the external stimulator 710 may apply high frequency stimulation pulses as described above with respect to FIG. 3. As a result of the applied bursts, transcutaneous current may flow between the source electrode 714 and the return electrode 716 through the skin 722 and the underlying subcutaneous tissue. During positive portions of the high frequency burst, current may flow from the source electrode 714 to the return electrode 716. During negative portions of the high frequency burst, current may flow from the return electrode 716 to the source electrode 714.

Some of the transcutaneous current passing between the source electrode 714 and the return electrode 716 may pass through the skin and the underlying tissue of the subject without passing through the implantable device 730. The implanted device 730 presents a path for current between the source electrode 714 and the return electrode 716, and a portion of the transcutaneous current passes between the source electrode 714 and the return electrode 716 through the implantable device 730.

During positive portions of a high frequency stimulation pulse, where current flows from the source electrode 714 to the return electrode 716, a portion of the transcutaneous current may pass through the first channel of the implantable device 730. The portion of the transcutaneous current may flow from the source electrode 714 to the receiving electrode 733 through a first tissue path 717. The current may flow from the receiving electrode 733 to the first output electrode 736 through the first channel, as the first rectification element of the first channel is configured to allow current to pass in that direction. The current may flow from the first output electrode 736 to the return electrode 716 through a second tissue path 718. The current passing along the second tissue path 718 may stimulate the nerve 724 at a location proximate the first output electrode 736 due to the proximity of the first output electrode 736 to the nerve 724. Little or no current may pass through the second channel, as the second rectification element is configured to inhibit current flowing from the receiving electrode 733 to the second output electrode 738.

During negative portions of a high frequency stimulation pulse, where current flows from the return electrode 716 to the source electrode 714, a portion of the transcutaneous current may pass through the second channel of the implantable device 730. The portion of the transcutaneous current may flow from the return electrode 716 to the second output electrode 738 through a third tissue path 719. The current may flow from the second output electrode 738 to the receiving electrode 733 through the second channel, as the second rectification element of the second channel is configured to allow current to pass in that direction. The current may flow from the receiving electrode 733 to the source electrode 714 through the first tissue path 717. The current passing along the third tissue path 719 may stimulate the nerve 724 at a location proximate the second output electrode 738 due to the proximity of the second output electrode 738 to the nerve 724. Little or no current may pass through the first channel, as the first rectification element is configured to inhibit current flowing from the first output electrode 736 to the receiving electrode 733.

In some aspects, the receiving electrode 733, the first output electrode 736, and the second output electrode 738 may have different shapes and sizes which may influence the density of current and stimulation charge relative to those electrodes. In some aspects, the receiving electrode 733 may have a larger surface area than the first output electrode 736 and/or the second output electrode 738. The larger surface area of the receiving electrode 733 may result in a lower current density and lower charge building in the tissue contacting the receiving electrode 733, reducing the threshold for transcutaneous current passing through the receiving electrode 733 to cause undesired activation or sensation at that location. The smaller surface area of the first output electrode 736 and/or the second output electrode 738 may result in a higher current density and higher charge building in the tissue contacting the first output electrode 736 and/or the second output electrode 738, lowering the threshold for desired activation (e.g., stimulation) at that location.

In some aspects, the implantable device may be implanted with the output electrodes in proximity to an afferent nerve (e.g., a sensory nerve). The output electrode coupled to the anodic channel of the implantable device may be implanted at a proximal location (e.g., closer to the brain of the subject) on the afferent nerve relative to the output electrode coupled to the cathodic channel. Current passing through the output electrode coupled to the cathodic channel may stimulate the afferent nerve and current passing through the output electrode coupled to the anodic channel may prevent signals from the stimulation from propagating to the brain and causing sensation. Alternatively, in some embodiments, an implantable device may include an output electrode coupled to the anodic channel implanted at a distal location on the afferent nerve relative to the output electrode coupled to the cathodic channel.

In some aspects, the implantable device may be implanted with the output electrodes in proximity to an efferent nerve (e.g., a motor nerve). The output electrode coupled to the anodic channel of the implantable device may be implanted at a distal location (e.g., further from the brain of the subject) on the efferent nerve relative to the output electrode coupled to the cathodic channel. Current passing through the output electrode coupled to the cathodic channel may stimulate the efferent nerve and current passing through the output electrode coupled to the anodic channel may prevent signals from the stimulation propagating to the muscles and causing a motor response. Alternatively, in some aspects, an implantable device may include an output electrode coupled to the anodic channel implanted at a proximal location on the efferent nerve relative to the output electrode coupled to the cathodic channel. In some aspects, any of the above described embodiments may be implanted with the electrodes in proximity to a nerve that includes both afferent and efferent fibers (e.g., a mixed nerve).

FIG. 8 is a circuit diagram of a multi-channel implantable device 800 for applying transcutaneous current. For example, the implantable device 800 may be the implantable device 730 described above with respect to FIG. 7. The implantable device 800 includes a receiving electrode 810, a first output electrode 838, and a second output electrode 828.

The implantable device 800 includes a first channel connecting the receiving electrode 810 with the first output electrode 838. The first channel includes a first diode 836, a first capacitor 832, and a first resistor 834. The implantable device 800 includes a second channel connecting the receiving electrode 810 with the second output electrode 828. The second channel includes a second diode 826, a second capacitor 822, and a second resistor 824.

The anode of the first diode 836 is coupled to the receiving electrode 810 and the cathode of the first diode 836 is coupled to the first output electrode 838. The first diode 836 will present a low impedance to current flowing from the receiving electrode 810 to the first output electrode 838 in the first channel. The first diode 836 will present a high impedance to current flowing from the first output electrode 838 to the receiving electrode 810.

The cathode of the second diode 826 is coupled to the receiving electrode 810 and the anode of the second diode 826 is coupled to the second output electrode 828. The second diode 826 will present a high impedance to current flowing from the receiving electrode 810 to the second output electrode 828 in the second channel. The second diode 826 will present a low impedance to current flowing from the second output electrode 828 to the receiving electrode 810.

In some aspects, the first diode 836 and/or the second diode 826 may be a Schottky diode.

The first capacitor 822 is connected between the receiving electrode 810 and the first diode 836. The second capacitor 832 is connected between the receiving electrode 810 and the second diode 824. The first capacitor 832 and the second capacitor 822 may serve a DC blocking function to ensure that the stimulation delivered to the nerve through their respective channels is charge balanced. The first capacitor 832 and the second capacitor 822 may have capacitances suitable to allow for the high frequency rectified current pulse to pass through with minimal attenuation or distortion, while allowing for the first capacitor 832 and the second capacitor 822 to adequately or fully discharge during the discharge periods between stimulation periods. For example, the capacitance of the first capacitor 832 and/or the second capacitor 822 may be on the order of several micro-Farads.

The first resistor 834 is connected in parallel with the first diode 836. The second resistor 834 is connected in parallel with the second diode 836. The first resistor 834 and the second resistor 824 may provide a path for the first capacitor 832 and the second capacitor 822 to discharge during the discharge periods. The first resistor 834 and the second resistor 824 may have resistances suitable to present high impedance relative to the first diode 836 and the second diode 826, such that little or no current flows through the first resistor 834 and the second resistor 824 during the stimulation periods, but to allow for the first capacitor 832 and the second capacitor 822 to adequately discharge during the discharge periods. For example, the resistance of the first resistor 834 and/or the second resistor 824 may be on the order of several kilo-Ohms.

In some aspects, a circuit for an implantable device may include more or fewer components than depicted in FIG. 8, or may include alternative components accomplishing the same or a similar function. For example, in some aspects, the circuit may include a transistor serving as the element rectifying the current instead of a diode. In some aspects, the circuit may not include the capacitor or the resistor. In some aspects, the circuit may include one or more element protecting against electrostatic discharge.

When an external generator delivers a high frequency transcutaneous burst to the implantable device 800 via the tissue surrounding the implantable device 800, the first diode 836 and the second diode 826 rectify the received electrical current. During positive portions of the high frequency burst, the current 850 at the first output electrode 838 is positive (e.g., flowing from the first output electrode 838 to the external generator electrode), as the first channel presents a low impedance pathway for current relative to the second channel and the surrounding tissue. During negative portions of the high frequency burst, the current 850 at the first output electrode 838 is zero, as the first channel presents a high impedance relative to the second channel. The current 850 at the first output electrode 838 therefore alternates between positive current and zero current. As the burst has a relatively high frequency, the electrical charge built during the individual pulses of the burst does not dissipate and a positive charge accumulates at the nerve membrane near the first output electrode 838.

During positive portions of the high frequency stimulation pulse, the current 860 at the second output electrode 828 is zero, as the second channel presents a high impedance relative to the first channel. During negative portions of the high frequency stimulation pulse, the current 860 at the second output electrode 828 is negative (e.g., flowing from the external generator electrode to the second output electrode 828), as the second channel presents a low impedance pathway for current relative to the first channel and the surrounding tissue. The current 860 at the second output electrode 828 therefore alternates between negative current and zero current. As the burst has a relatively high frequency, the electrical charge built during the individual pulses of the burst does not dissipate and a negative charge accumulates at the nerve membrane near the second output electrode 828.

The current of the first channel and of the second channel both flow through the receiving electrode 810. The current 840 at the receiving electrode 810 includes both the positive portions of the first channel current and the negative portions of the second channel current. As the current 840 includes both the positive portions of the first channel current and the negative portions of the second channel current, charge may not accumulate in the tissue surrounding the receiving electrode 810.

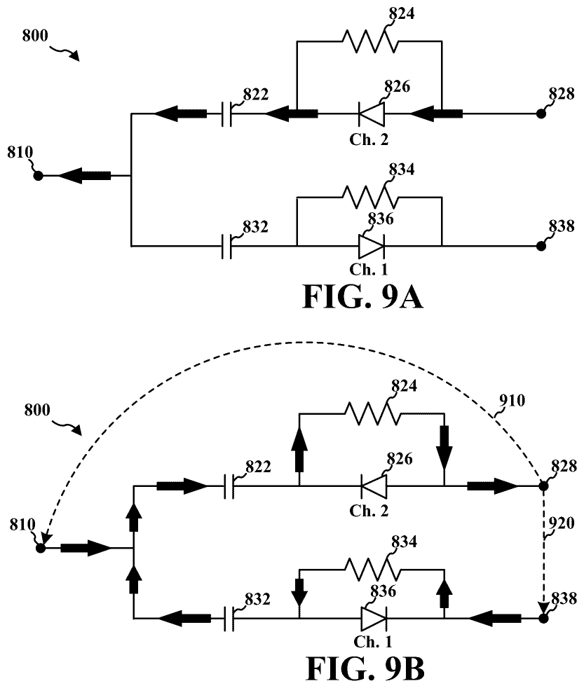

FIG. 9A is a circuit diagram illustrating current flow in the second channel of the implantable device 800 of FIG. 8 during the burst phase of the transcutaneous current. For example, FIG. 9A may illustrate the current flow during the burst 322 with respect to FIG. 3. The arrows represent the flow of current in the second channel. The flow of current for the first channel is not shown.

During the negative portions of the transcutaneous bust, current flows from the second output electrode 828, through the second diode 826, and through the second capacitor 822, to the receiving electrode 810. Over the duration of the burst, the repeated individual pulses of negative current charge the second capacitor 822.

FIG. 9B is a circuit diagram illustrating current flow in the implantable device 800 of FIG. 8 during the discharge phase of transcutaneous stimulation due to the discharging of the second channel capacitor. Current flow due to the discharge of the first channel capacitor is not shown.

In the second channel the discharge current flows from the second capacitor 822, which was charged during the burst phase. The discharge current flows through the second resistor 824 to the second output electrode 828. A portion of the discharge current flows via a first return path 910 through the surrounding tissue to the receiving electrode 810, and back to the second capacitor 822. Another portion of the discharge current flows via a second return path 920 through the surrounding tissue to the first output electrode 838, through the first resistor 834, through the first capacitor 832, and back to the second capacitor 822.

The implantable device 800 being configured to allow discharge current to return via the first return path 910 and the second return path 920 may allow the second capacitor 822 to discharge faster, relative to a single channel embodiment, such as the embodiment described above with respect to FIG. 5, which may allow for the implantable device to deliver stimulation current more efficiently and/or may allow for a smaller capacitor, reducing the size of the implantable device. Faster discharge of the capacitor may also allow for shorter time between the bursts and thus may allow for the application higher burst repetition frequency.

Although not shown, the first capacitor 832 may similarly discharge via two paths, a first from the first output electrode 838 through the surrounding tissue to the receiving electrode 810 and a second from the first output electrode 838 through the surrounding tissue to the second output electrode 828. The first capacitor 832 may similarly discharge faster relative to a single channel embodiment.

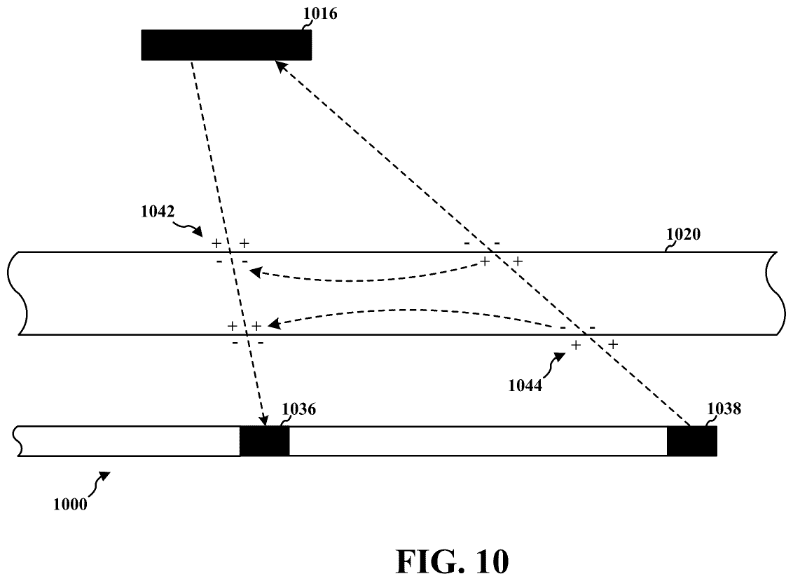

FIG. 10 is a diagram illustrating stimulation of a nerve utilizing monopolar and quasi-bipolar stimulation. An implantable device 1000 is implanted beneath the skin of a subject. For example, the implantable device 1000 may be the implantable device 730 described above with respect to FIG. 7. The first output electrode 1036 and the second output electrode 1038 are implanted in proximity to a nerve 1020 of the subject. A return electrode 1016 of an external generator is placed on the skin of the subject.

Monopolar stimulation may refer to stimulation caused by stimulating current which flows from the output electrode to a distant return electrode not located near the nerve (e.g., the return electrode 1016). Bipolar stimulation may refer to stimulation caused by stimulating current which flows between the two output electrodes located near the targeted nerve. Quasi-bipolar stimulation may refer to stimulation generated by the adjacent anodic and cathodic channels, which together cause a pattern of the electrical field similar to that of the bipolar stimulation without having stimulating current flow between the two output electrodes.

A first channel of the implantable device 1000 connected to the first output electrode 1036 may be configured for cathodic stimulation. When an alternating transcutaneous current is applied by the external generator, current may flow into the first output electrode 1036 from the return electrode 1016, and may flow through the nerve 1020 at a first position 1042. In a resting state, the outer surface of the nerve 1020 (e.g., the nerve membrane) may have a higher electrical potential than the inside of the nerve 1020. The current flowing into the first output electrode 1036 from the return electrode 1016 may cause a decrease in the difference of the electrical potential across parts of the surface of the nerve, which may cause the nerve membrane to depolarize and trigger the nerve to fire via monopolar stimulation of the nerve. Depending on the geometrical orientation between the electrode 1036 and the nerve, the current flowing into the first output electrode 1036 from the return electrode 1016 may increase the difference in the electrical potential across different locations of the nerve surface, thus causing hyperpolarization.

A second channel of the implantable device 1000 connected to the second output electrode 1038 may be configured for anodic stimulation. When an alternating transcutaneous current is applied by the external generator, current may flow into the return electrode 1016 from the second output electrode 1038, and may flow through the nerve 1020 at a second position 1044. The current flowing from the second output electrode 1038 to the return electrode 1016 may cause a decrease in the difference of the electrical potential across parts of the surface of the nerve, which may cause the nerve membrane to depolarize and trigger the nerve to fire via monopolar stimulation of the nerve. Depending on the geometrical orientation between the electrode 1038 and the nerve, the current flowing from the second output electrode 1036 to the return electrode 1016 may increase the difference in the electrical potential across different locations of the nerve surface, thus causing hyperpolarization.

The current flowing into the first output electrode 1036 from the return electrode 1016 may decrease the potential of the inside of the nerve relative to the surface of the nerve at some portions of the first position 1042 of the nerve 1020 and it may increase the potential of the nerve relative to the surface in other portions of the nerve. The current flowing from the second output electrode 1038 into the return electrode 1016 may decrease the potential of the inside of the nerve relative to the surface of the nerve at some portions of the first position 1044 of the nerve 1020 and it may increase the potential of the nerve relative to the surface in other portions of the nerve. The increased and decreased potentials may result in an electrical field between the first position 1042 and the second position 1044, which may result in a pattern of the electrical field along the nerve similar to that of the bipolar stimulation of the nerve 1020.

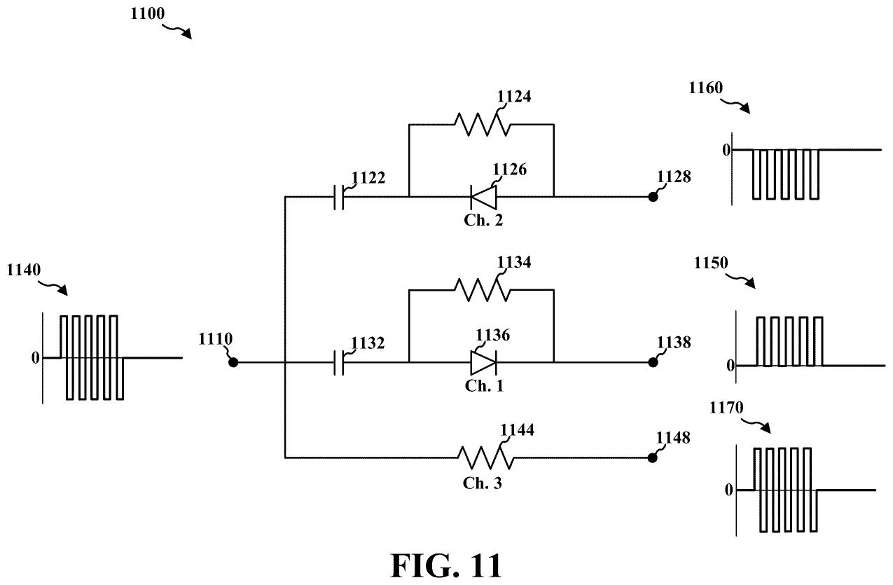

FIG. 11 is a circuit diagram of an implantable device 1100 including two channels and a bypass channel. The implantable device 1100 includes a receiving electrode 1110, a first output electrode 1138, a second output electrode 1128, and a bypass electrode 1148. The first channel, between the receiving electrode 1110 and the first output electrode 1138, includes a first diode 1130 oriented for anodic stimulation (e.g., to allow current to pass from the receiving electrode 1110 to the first output electrode 1138). The second channel, between the receiving electrode 1110 and the second output electrode 1128, includes a second diode 1126 oriented for cathodic stimulation (e.g., to allow current to pass from the second output electrode 1128 to the receiving electrode 1110).

The bypass channel, between the receiving electrode 1110 and the bypass electrode 1148, includes a resistor 1144. The resistor 1144 may have a resistance that results in the bypass channel having a path resistance similar to the first stimulating channel and the second stimulating channel. For example, the resistor 1144 may have a resistance that is the same or similar to the resistance of the first diode 1126 and the second diode 1136 when in a conductive state.

When the implantable device 1100 is exposed to a transcutaneous alternating current, the first diode 1136 and the second diode 1126 rectify the current passing through their respective channels. During positive portions of the transcutaneous alternating current, the positive current passes through the first channel and the bypass channel. During negative portions of the transcutaneous alternating current, the negative current passes through the second channel and the bypass channel. The current 1140 at the receiving electrode 1110 will include both the positive and the negative portions of the transcutaneous alternating current. The current 1150 at the first output electrode 1138 will include the positive portions of the transcutaneous alternating current but not the negative portions (e.g., will alternate between positive current and no current). The current 1160 at the second output electrode 1128 will include the negative portions of the transcutaneous alternating current but not the positive portions (e.g., will alternate between negative current and no current. The current 1170 at the bypass electrode 1148 will include both the positive and the negative portions of the transcutaneous alternating current.

The alternating current applied to the nerve by the bypass electrode 1148 may prevent signals from propagating through the portion of the nerve proximate the bypass electrode 1148. The bypass electrode 1148 may be located relative to the first output electrode 1138 and the second output electrode 1128 such that the alternating current applied by the bypass electrode 1148 prevents signals caused by the stimulation applied to the nerve by the first output electrode 1138 or the second output electrode 1128 from propagating beyond the bypass electrode 1148. For example, in an efferent nerve sending signals to a muscle of the subject, the bypass electrode 1128 may be located distally relative to the first output electrode 1138 and the second output electrode 1128 and may prevent stimulation at the first output electrode 1138 and the second output electrode 1128 from causing unwanted contractions in the muscle. In an afferent nerve sending sensory signals to the brain, the bypass electrode 1128 may be located proximally relative to the first output electrode 1138 and the second output electrode 1128 and may prevent stimulation at the first output electrode 1138 and the second output electrode 1128 from causing perceived pain.

The bypass channel may also deliver direct current or low frequency current at the bypass electrode 1148. When the implantable device 1100 is exposed to a direct transcutaneous current, or to an alternating transcutaneous current at a low enough frequency, the first capacitor 1132 and/or the second capacitor 1122 will fully charge, preventing current from flowing through their respective channels. The transcutaneous current will still be able to pass through the bypass channel and stimulate the nerve at the bypass electrode 1148.

In some aspects, an implantable device may include a bypass channel as described above with only one channel (e.g., a bypass channel and an anodic channel, or a bypass channel and a cathodic channel). In some aspects, an implantable device may include a bypass channel with more than two channels (e.g., a bypass channel, an anodic channel, and two cathodic channels).

In some aspects, an implantable device may be implanted with the output electrodes proximate an efferent nerve. The implantable device may include a bypass channel coupled to a bypass electrode, and the bypass electrode may be between the output electrodes and the receiving electrode. In some aspects, an implantable device may be implanted with the output electrodes proximate an afferent nerve. The implantable device may include a bypass channel coupled to a bypass electrode, and the output electrodes may be between the bypass electrode and the receiving electrode. In some aspects, an implantable device may be implanted with the output electrodes proximate a mixed nerve (e.g., a nerve containing both afferent and efferent fibers). The implantable device may include a bypass channel coupled to a bypass electrode, and the bypass electrode may be between the first output electrode and the second output electrode.

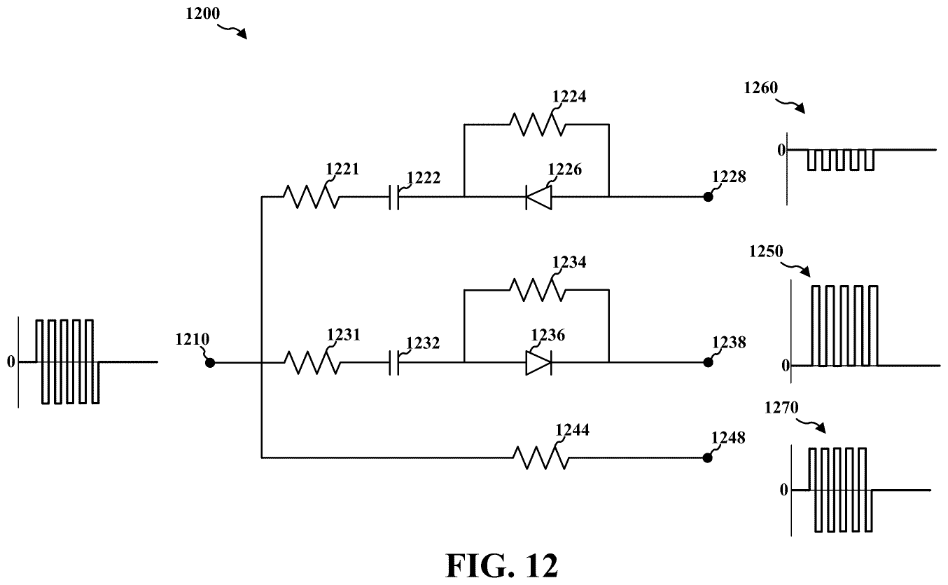

FIG. 12 is a circuit diagram of an implantable device 1200 including two channels and a bypass channel. The implantable device 1200 includes a receiving electrode 1210, a first output electrode 1238, a second output electrode 1228, and a bypass electrode 1248. The first channel, between the receiving electrode 1210 and the first output electrode 1238, includes a first diode 1236 oriented for anodic stimulation (e.g., to allow current to pass from the receiving electrode 1210 to the first output electrode 1238) and a first resistor 1231 in series with the first diode 1236. The second channel, between the receiving electrode 1210 and the second output electrode 1228, includes a second diode 1226 oriented for cathodic stimulation (e.g., to allow current to pass from the second output electrode 1228 to the receiving electrode 1210) and a second resistor 1221 in series with the second diode 1226. The bypass channel, between the receiving electrode 1110 and the bypass electrode 1148, includes a third resistor 1144.

When the implantable device receives portion of the transcutaneous alternating current, the resistances of the first resistor 1231, the second resistor 1221, and the third resistor 1144 determine how much current passes through their respective channels. It is possible to measure the resistance of each channel of the implant before the implantable device 1200 is implanted. 1231 before the implantation. It is thus possible to adjust the resistance of the resistors 1221 and 1231 or to select the resistors 1221 and 1231 to achieve a specific ratio between the resistances of the channels. The ratio between the resistances of the channels will determine the ratio between the amplitude of the currents delivered by these channels. For example, as illustrated in FIG. 12, the first resistor 1231 may have a low resistance relative to the second resistor 1221. The first channel may allow current to pass during positive portions of the transcutaneous alternating current, and the current 1250 at the first output electrode 1238 may alternate between positive current and zero current. The second channel may allow current to pass during negative portions of the transcutaneous alternating current, and the current 1260 at the second output electrode 1228 may alternate between negative current and zero current. Because the first resistor 1231 has a lower resistance, the amplitude of the current at the first output electrode 1238 may be higher and may cause higher intensity stimulation at the first output electrode 1238. Because the second resistor 1221 has a higher resistance, the amplitude of the current at the second output electrode 1228 may be lower and may cause lower intensity stimulation at the second output electrode 1238.

In some aspects, t the resistor 1244 of the bypass channel has a resistance such that the resistance of the bypass channel is lower than the overall resistance of the first channel in its conductive state (via the resistor 1221, the capacitor 1222, and the diode 1226) and higher than the overall resistance of the second channel in its conductive state (via the resistor 1231, the capacitor 1232, and the diode 1236). Thus the negative amplitude of the current passing through the bypass channel as shown in 1270 is higher than the negative amplitude of the first channel as shown in 1260, and the positive amplitude of the bypass channel is lower than the positive amplitude of the second channel as shown in 1250.

In some aspects, instead of the first resistor 1231, the second resistor 1221, and the third resistor 1244, other impedance elements may be used. For example, one or more may be replaced with a capacitor or an inductor, and the impedance of the capacitor or the inductor may impact the current in that channel as described above with respect to a resistor.

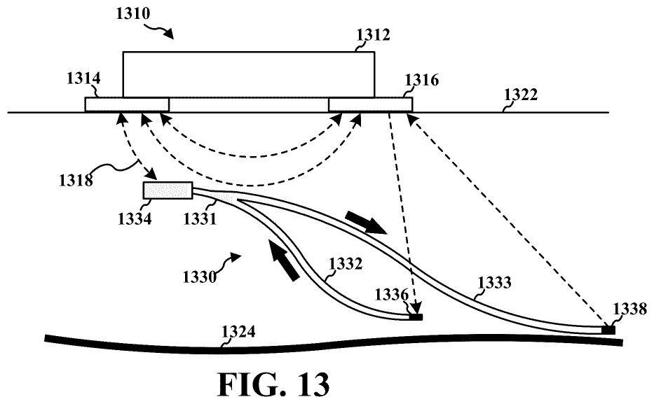

FIG. 13 is a diagram illustrating a system for applying multi-channel transcutaneous current utilizing a branched lead. The system includes an external generator 1310 and an implantable device 1330. The external generator 1310 may function as described above with respect to the external generator 710 of FIG. 7. The implantable device 1330 includes a receiver 1334, a lead 1331, a first output electrode 1336, and a second output electrode 1338. The lead 1331 includes a first branch 1332 and a second branch 1333. The first output electrode 1336 is on the first branch 1332 of the lead 1331 and the second output electrode 1338 is on the second branch 1333 of the lead 1331. The implantable device 1330 may otherwise function as described above with respect to the implantable device 730 of FIG. 7.

In some aspects, instead of a branched lead, an implantable device may include two leads. Each lead may include an output electrode at one end and may be fixed to the receiver at the other end, and the implantable device may otherwise function as described above with respect to the implantable device 730 of FIG. 7.

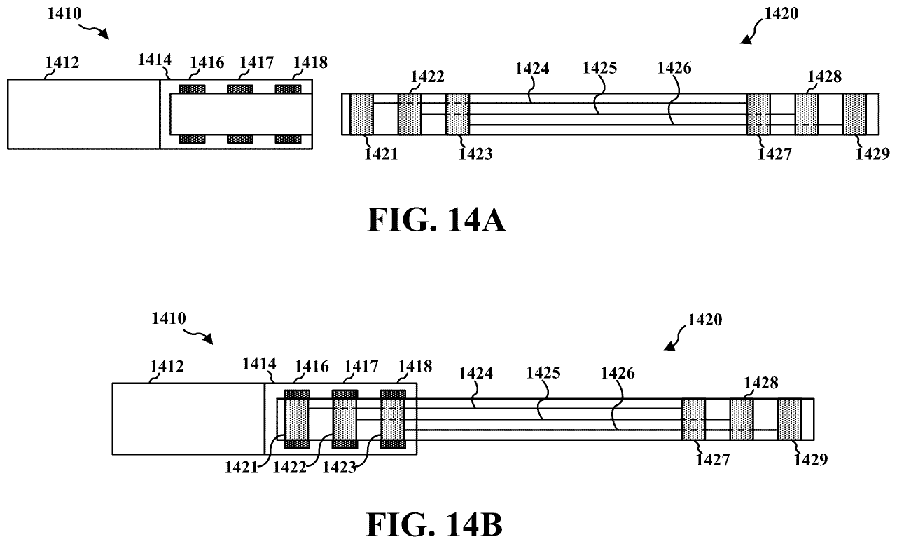

FIGS. 14A and 14B illustrate a configuration for an implantable device with a separable receiver 1410 and lead 1420. FIG. 14A is a diagram illustrating the implantable device with the receiver 1410 and the lead 1420 in a separated state. FIG. 14B is a diagram illustrating the implantable device with the lead 1420 coupled to the receiver 1410.

The receiver 1410 includes an electronics housing 1412 and a lead connector 1414. Electronic components, such as rectification elements for each channel of the implantable device, may be within the electronics housing 1412. The electronics housing 1412 may include an outer shell hermetically sealed to the lead connector 1414 to form a hermetically sealed cavity and the electronic components may be within the hermetically sealed cavity. The receiver 1410 may also include a receiving electrode. For example, some or all of the outer shell of the electronics housing 1412 may be metal and may be utilized as the receiving electrode.

The lead connector 1414 may include electrodes for electrically coupling with electrodes on the lead 1420 when the lead 1420 is coupled to the lead connector 1410. For example, as illustrated in FIGS. 14A and 14B, the lead connector 1414 includes a first electrode 1416, a second electrode 1417, and a third electrode 1418. The electrodes of the lead connector 1414 may be electrically coupled to the electronics in the electronics housing 1412 via hermetic feedthroughs between the electronics housing 1412 and the lead connector 1414. The lead connector 1414 may include an engagement component such as a lead screw (not shown) to mechanically fix the lead 1420 into the lead connector 1414.

The lead 1420 has a first end configured to be fixed into the lead connector 1414, hereinafter the proximal end, and a second end configured to be implanted, exposed, at a location where the implantable device will deliver transcutaneous current, hereinafter the distal end. The lead 1420 includes a first set of electrodes at or near the proximal end, which are located within the lead connector 1414 when the lead 1420 is coupled to the lead connector 1414. For example, as illustrated in FIGS. 14A and 14B, the lead 1420 includes a first electrode 1421, a second electrode 1422, and a third electrode 1423. When the lead 1420 is fixed into the lead connector 1414, the first set of electrodes of the lead 1420 are in electrical contact with the electrodes of the lead connector 1414.

The lead 1420 includes a second set of electrodes which may be exposed when the lead 1420 is coupled to the lead connector 1414. For example, as illustrated in FIGS. 14A and 14B, the lead 1420 includes a fourth electrode 1427, a fifth electrode 1428, and a sixth electrode 1429. In some aspect, the second set of electrodes may be at or near the distal end of the lead 1420. In some aspects, some or all of the second set of electrodes may be at medial locations between the proximal and distal ends of the lead 1420.

The lead 1420 may include insulated conductors inside the lead 1520 coupling the first set of electrodes to the second set of electrodes. For example, as illustrated in FIGS. 14A and 14B, the lead 1420 includes a first insulated conductor 1424 coupling the first electrode 1421 to the fourth electrode 1427, a second insulated conductor 1425 coupling the second electrode 1422 to the fifth electrode 1428, and a third insulated conductor 1426 coupling the third electrode 1423 to the sixth electrode 1429. In some aspects, each electrode in the second set of electrodes may be coupled to only one electrode of the first set of electrodes. In some aspects, as illustrated in FIGS. 14A and 14B, each electrode of the first set of electrodes may be coupled to one corresponding electrode in the second set of electrodes. In some aspects, multiple electrodes in the second set of electrodes may be coupled to a single electrode in the first set of electrodes (e.g., the lead 1420 may include an insulated conductor that couples one electrode in the first set of electrodes to multiple electrodes at exposed portions of the lead 1420).

When the lead 1420 is fixed into the lead connector 1414, complete channels (e.g., channels, bypass channels) are formed between the receiving electrode and the second set of electrodes. For example, the implantable device of FIGS. 14A and 14B includes three channels. A first channel between the receiving electrode and the fourth electrode 1427 runs from the receiving electrode of the receiver 1410, through any electronic components for the first channel (e.g., a rectifier) in the electronics housing 1412, to the first electrode 1416 of the receiver 1410, to the first electrode 1421 of the lead 1420, through the first insulated conductor 1424 of the lead, to the fourth electrode 1427 of the lead 1420. A second channel between the receiving electrode and the fifth electrode 1426 runs from the receiving electrode of the receiver 1410, through any electronic components for the second channel (e.g., a rectifier) in the electronics housing 1412, to the second electrode 1417 of the receiver 1410, to the second electrode 1422 of the lead 1420, through the second insulated conductor 1425 of the lead, to the fifth electrode 1428 of the lead 1420. A third channel between the receiving electrode and the sixth electrode 1427 runs from the receiving electrode of the receiver 1410, through any electronic components for the third channel (e.g., a rectifier) in the electronics housing 1412, to the third electrode 1418 of the receiver 1410, to the third electrode 1423 of the lead 1420, through the third insulated conductor 1426 of the lead, to the sixth electrode 1429 of the lead 1420.

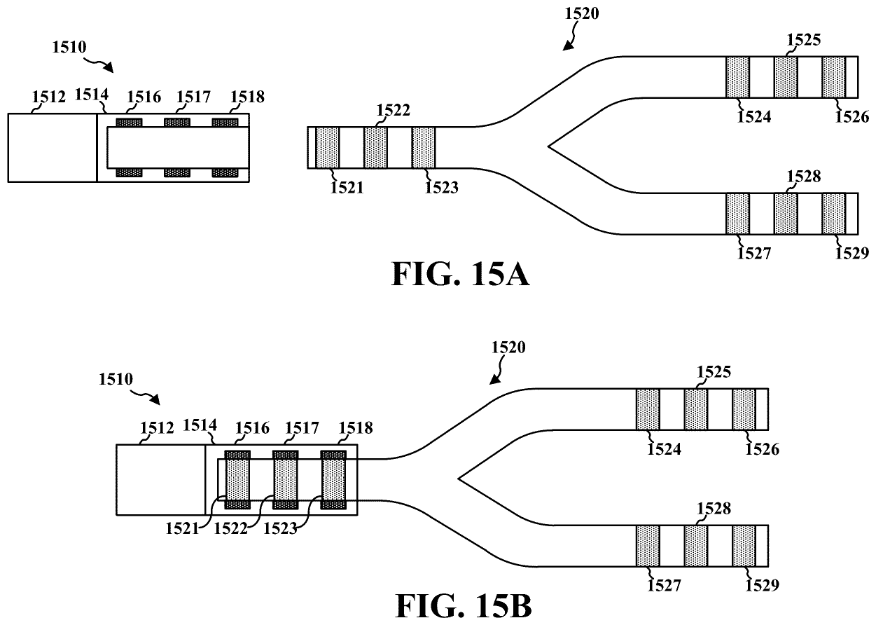

FIGS. 15A and 15B illustrate a configuration for an implantable device with a branched lead 1520 and separable receiver 1510. FIG. 15A is a diagram illustrating the implantable device with the receiver 1510 and the branched lead 1520 in a separated state. FIG. 15B is a diagram illustrating the implantable device with the branched lead 1520 coupled to the receiver 1510.

The receiver 1510 includes an electronics housing 1512 and a lead connector 1514. The lead connector 1514 may include a first electrode 1516, a second electrode 1517, and a third electrode 1518. The receiver 1510 may be configured as described above with respect to the receiver 1410 of FIGS. 14A and 14B.

The lead 1520 has a first end configured to be fixed into the lead connector 1514, hereinafter the proximal end. The lead 1520 includes a first set of electrodes at or near the proximal end, which are located within the lead connector 1514 when the lead 1520 is coupled to the lead connector 1514. For example, as illustrated in FIGS. 15A and 15B, the lead 1520 includes a first electrode 1521, a second electrode 1522, and a third electrode 1523. When the lead 1520 is fixed into the lead connector 1514, the first set of electrodes of the lead 1520 are in electrical contact with the electrodes of the lead connector 1514.

The lead 1520 includes two branches. The end of the first branch will be referred to hereinafter as the first distal end of the lead 1520 and the end of the second branch will be referred to hereinafter as the second distal end of the lead 1520. The first and second distal ends of the leads may be configured to be implanted, exposed, at locations where the implantable device will deliver transcutaneous current. The lead 1520 includes a second set of electrodes at the first distal end of the lead 1520. For example, as illustrated in FIGS. 15A and 15B, the second set of electrodes includes a fourth electrode 1524, a fifth electrode 1525, and a sixth electrode 1526. The lead 1520 includes a third set of electrodes at the second distal end of the lead 1520. For example, as illustrated in FIGS. 15A and 15B, the third set of electrodes includes a seventh electrode 1527, an eighth electrode 1528, and a ninth electrode 1529.

The lead 1520 may include insulated conductors inside the lead 1520 coupling some or all of the first set of electrodes to the second set of electrodes and coupling some or all of the first set of electrodes to the third set of electrodes. When the lead 1520 is fixed into the lead connector 1514, complete channels (e.g., channels, bypass channels) are formed between the receiving electrode and the electrodes of the second and third sets of electrodes.

In some aspects, each electrode of the first set of electrodes may be coupled to an electrode in each branch of the lead 1520. For example, the first electrode 1521 may be coupled to both the fourth electrode 1524 and to the seventh electrode 1527, forming a first channel between the receiving electrode and the fourth electrode 1524 and the seventh electrode 1527. The second electrode 1522 may be coupled to both the fifth electrode 1525 and to the eighth electrode 1528, forming a second channel between the receiving electrode and the fifth electrode 1525 and the eighth electrode 1528. The third electrode 1523 may be coupled to both the sixth electrode 1526 and the ninth electrode 1529, forming a third channel between the receiving electrode and the sixth electrode 1526 and the ninth electrode 1529.

In some aspects, one or more electrode of the first set of electrodes may be coupled to electrodes in only one branch of the lead 1520. For example, the first electrode 1521 may be coupled to the fourth electrode 1524 and the fifth electrode 1524, forming a first channel. The first channel may be a channel configured to stimulate tissue proximate the first distal end of the lead 1520. The second electrode 1522 may be coupled to the seventh electrode 1527 and the eighth electrode 1528, forming a second channel. The second channel may be a channel configured to stimulate tissue proximate the second distal end of the lead 1520. The first channel may include a rectifier in the electronics housing 1512 allowing current to pass in the anodic direction, and the second channel may include a rectifier in the electronics housing 1512 allowing current to pass in the cathodic direction. The third electrode 1523 may be coupled to the sixth electrode 1526 and the ninth electrode 1529, forming a third channel. The third channel may be a high-frequency bypass channel for both the first distal end of the lead and the second distal end of the lead as described above with respect to FIG. 11.

In some aspects, the lead 1520 may include electrodes in other positions on the lead 1520. For example, one or more electrode in the second set of electrodes or the third set of electrodes may be located toward the proximal end of the lead 1520, before the lead 1520 branches into the first branch and the second branch. In some aspects, a lead according to the present disclosure may include more than two branches.

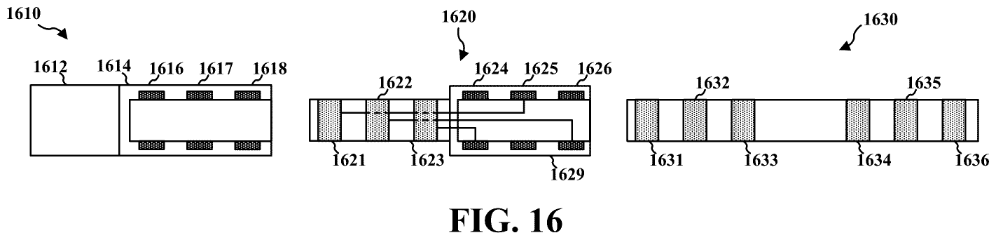

FIG. 16 is a diagram illustrating an implantable device utilizing an adapter 1620. The implantable device includes a receiver 1610, an adapter 1620, and a lead 1630. The implantable device is depicted with the receiver 1610, the adapter 1620, and the lead 1630 in a separated state. The receiver 1610 may be fixed to the adapter 1620 and the adapter 1630 may be fixed to the lead 1630 to form the implantable device in a connected state.

The receiver 1610 includes an electronics housing 1612 and a lead connector 1614. The lead connector 1614 may include a first electrode 1616, a second electrode 1617, and a third electrode 1618. The receiver 1610 may be configured as described above with respect to the receiver 1410 of FIGS. 14A and 14B.

The lead 1630 has a first end configured to be fixed into the adapter 1630, hereinafter the proximal end. The lead 1630 includes a first set of electrodes at or near the proximal end. The first set of electrodes includes a first electrode 1631, a second electrode 1632, and a third electrode 1633. The first set of electrodes are positioned on the lead 1630 such that they are located within the adapter 1620 when the lead 1630 is fixed into the adapter 1620. The lead 1630 has a second end which is referred to hereinafter as the distal end. The lead 1630 includes a second set of electrodes which may be at or near the distal end of the lead 1630. The second set of electrodes includes a fourth electrode 1634, a fifth electrode 1635, and a sixth electrode 1636. The second set of electrodes may be positioned on the lead 1630 such that they are exposed when the lead 1630 is fixed into the adapter 1620. The lead 1630 may include insulated conductors coupling the electrodes of the first set of electrodes to the electrodes of the second set of electrodes, for example as described above with respect to the lead 1420 of FIGS. 14A and 14B.

The adapter 1620 includes a first set of electrodes and a second set of electrodes. The first set of electrodes includes a first electrode 1621, a second electrode 1622, and a third electrode 1623. The second set of electrodes includes a fourth electrode 1624, a fifth electrode 1625, and a sixth electrode 1626. The first set of electrodes are on a portion of the adapter 1620 which may be inserted and fixed into the lead connector 1614 of the receiver 1610. The first set of electrodes are positioned on the adapter 1620 such that they are located within the lead connector 1614 when the adapter 1620 is fixed into the lead connector 1614. The adapter 1620 includes a lead connector 1629. The second set of electrodes are on the lead connector 1629. The first set of electrodes are electrically coupled to the second set of electrodes. For example, as illustrated in FIG. 16, the first electrode 1621 is electrically coupled to the fifth electrode 1625, the second electrode 1622 is electrically coupled to the sixth electrode 1626, and the third electrode 1623 is electrically coupled to the fourth electrode 1624.

When the lead 1630 is fixed into the adapter 1620 and the adapter 1620 is fixed into the receiver 1610, complete channels are formed between the receiving electrode and the second set of electrodes of the lead 1630. For example, in the implantable device of FIG. 16, a channel between the receiving electrode and the fifth electrode 1635 runs from the receiving electrode of the receiver 1610, through any electronic components for the first channel (e.g., a rectifier) in the electronics housing 1612, to the first electrode 1616 of the receiver 1610, to the first electrode 1621 of the adapter 1620, to the fifth electrode 1625 of the adapter 1620, to the second electrode of the lead 1630, to the fifth electrode of the lead 1630.

The configuration of the connections between the first set of electrodes of the adapter 1620 and the second set of electrodes of the adapter 1620 determines which electrode of the lead 1630 is coupled to a channel. Adapters with different configurations can be utilized to realize different channel configurations in the same receivers and leads. For example, the receiver 1610 may include an anodic rectifier coupled to the first electrode 1616, a cathodic rectifier coupled to the second electrode 1617, and no circuitry coupled to the third electrode 1618. The adapter 1620 of FIG. 16 would result in the fourth electrode 1634 of the lead 1630 being coupled to the bypass channel, the fifth electrode 1635 being coupled to the anodic channel, and the sixth electrode 1636 being coupled to the cathodic channel. Another adapter may have the first electrode 1621 coupled to the sixth electrode 1626, the second electrode 1622 coupled to the fifth electrode 1625, and the third electrode coupled to the fourth electrode 1624. An implantable device using such an adapter would have the fourth electrode 1634 of the lead 1630 coupled to the bypass channel, the fifth electrode 1635 being coupled to the cathodic channel, and the sixth electrode 1636 being coupled to the anodic channel.

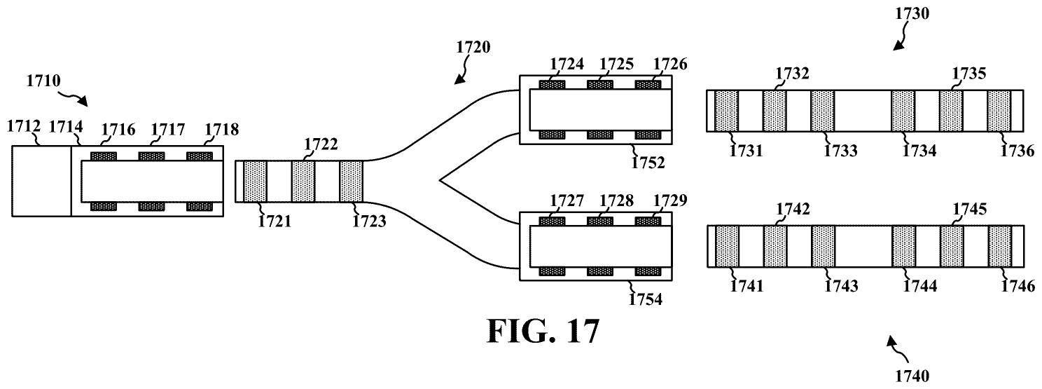

FIG. 17 is a diagram illustrating an implantable device utilizing a branched adapter 1720. The implantable device includes a receiver 1710, a branched adapter 1720, a first lead 1730, and a second lead 1740. The implantable device is depicted with the receiver 1720, the branched adapter 1720, the first lead 1730, and the second lead 1740 in a separated state. The receiver 1710 may be fixed to the branched adapter 1720, the first lead 1730 may be fixed to the branched adapter 1720, and the second lead 1740 may be fixed to the branched adapter 1720 to form the implantable device in a connected state.

The receiver 1710 includes an electronics housing 1712 and a lead connector 1714. The lead connector 1714 may include a first electrode 1716, a second electrode 1717, and a third electrode 1718. The receiver 1710 may be configured as described above with respect to the receiver 1410 of FIGS. 14A and 14B.

Both the first lead 1730 and the second lead 1740 include a first end configured to be fixed into the branched adapter 1720, hereinafter referred to as the proximal end, and includes a first set of electrodes positioned such that they are located within the branched adapter 1720 when the lead is fixed into the branched adapter 1720. The first set of electrodes of the first lead 1730 includes a first electrode 1731, a second electrode 1732, and a third electrode 1733. The first set of electrodes of the second lead 1740 includes a first electrode 1741, a second electrode 1742, and a third electrode 1743. Both the first lead 1730 and the second lead 1730 include a second end, hereinafter the distal end, and include a second set of electrodes positioned on the lead such that they are exposed when the lead is fixed into the branched adapter 1720. The second set of electrodes of the first lead 1730 includes a fourth electrode 1734, a fifth electrode 1735, and a sixth electrode 1736. The second set of electrodes of the second lead 1740 includes a fourth electrode 1744, a fifth electrode 1745, and a sixth electrode 1746. The first lead 1730 and the second lead 1740 may be configured as described above with respect to the lead 1430 of FIGS. 14A and 14B.

The branched adapter 1720 includes a first end configured to be coupled into the lead connector 1714 of the receiver 1710.

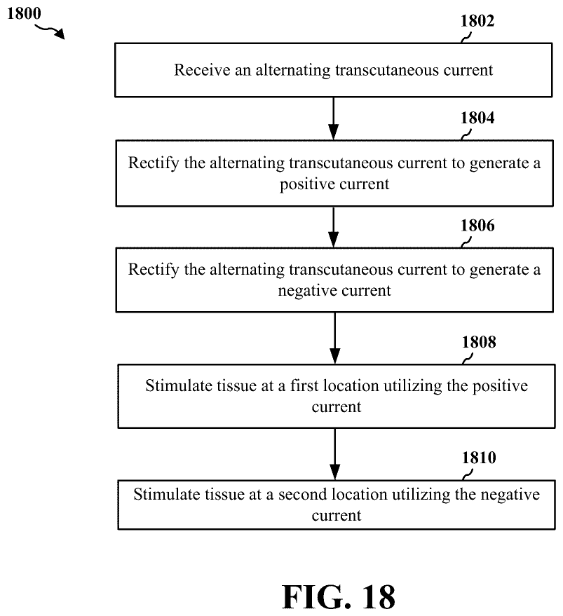

FIG. 18 is a flowchart 1800 illustrating a method of applying current to a tissue of a subject. The method may be performed at by an implantable device (e.g., the implantable device 730, 800, 1000, 1100, 1200, 1330).

At 1802, the implantable device receives an alternating transcutaneous current. The alternating transcutaneous current may be received from an external generator. The alternating transcutaneous current received by the implantable device may be a fraction of a current applied by the external generator. The alternating transcutaneous current may include a positive portion and a negative portion. The alternating transcutaneous current may be a series of bursts of alternating transcutaneous current The alternating transcutaneous current may be a series of bursts of alternating transcutaneous current, and the positive portion of the alternating transcutaneous current may be a positive portion of the series of bursts of alternating transcutaneous current, and the negative portion of the transcutaneous current may be a negative portion of the series of bursts of alternating transcutaneous current. The alternating transcutaneous current may be received through electrically conductive coupling. The electrically conductive coupling may be electrically coupling through conductive body tissue.

In some aspects, the alternating transcutaneous current may be a high frequency alternating transcutaneous current. In some aspects, the alternating transcutaneous current may be a burst of alternating transcutaneous current with a carrier frequency between 5 kHz and 100 kHz. In some aspects, the alternating transcutaneous current may be a burst of alternating transcutaneous current with a carrier frequency between 100 kHz and 5 MHz. In some aspects, the alternating transcutaneous current may be a burst of alternating transcutaneous current with a carrier frequency between 1 kHz and 5 kHz.

In some aspects, the alternating transcutaneous current may be a burst of alternating transcutaneous current and the carrier frequency of the alternating transcutaneous current may have a first value at a first time during the burst of alternating transcutaneous current and may have a second value at a second time during the burst of alternating transcutaneous current, the first value being different than the second value. In some aspects, the transcutaneous current may include a first burst of alternating transcutaneous current and a second burst of alternating transcutaneous current, and a carrier frequency of the transcutaneous current during the first burst may be different than a carrier frequency of the transcutaneous current during the second burst. In some aspects, the transcutaneous current may include a first burst of alternating transcutaneous current and a second burst of alternating transcutaneous current, and a carrier waveform of the transcutaneous current during the first burst may be different than a carrier waveform of the transcutaneous current during the second burst.

At 1804, the implantable device rectifies the alternating transcutaneous current received at 1802 to generate a positive current. The implantable device may include a first channel configured to allow the positive portion of the alternating transcutaneous current to pass from a receiving electrode of the implantable device to a first output electrode of the implantable device and to inhibit the negative portion of the alternating transcutaneous current passing from the first output electrode to the receiving electrode, the first output electrode being at the first location. The first output electrode may be implanted in proximity to a nerve of a subject (e.g., the first location), and the receiving electrode may be implanted in proximity to a surface of the skin of the subject. The alternating transcutaneous current may be a high frequency alternating transcutaneous current and the positive current may be a positive high frequency current.

At 1806 the implantable device rectifies the alternating transcutaneous current received at 1802 to generate a negative current. The implantable device may include a second channel configured to allow the negative portion of the transcutaneous current to pass from a second output electrode of the implantable device to the receiving electrode and to inhibit the positive portion of the transcutaneous current passing from the receiving electrode to the second output electrode. The second output electrode may be implanted in proximity to a nerve of a subject (e.g., the second location), and the receiving electrode may be implanted in proximity to a surface of the skin of the subject. The alternating transcutaneous current may be a high frequency alternating transcutaneous current and the negative current may be a negative high frequency current.

At 1808, the implantable device stimulates tissue at a first location utilizing the positive current from 1804.

At 1810, the implantable device stimulates tissue at a second location utilizing the negative current from 1806.

In some aspects, stimulating tissue at the first location may include applying monopolar stimulation to the tissue at the first location, stimulating tissue at the second location may include applying monopolar stimulation to the tissue at the second location, and the implantable device may apply quasi-bipolar stimulation between the first location and the second location based on applying monopolar stimulation to the tissue at the first location and applying monopolar stimulation at the second location.

In some aspects, the implantable device may apply an unrectified current to tissue at a third location. The implantable device may include a bypass channel configured to allow transcutaneous current to pass between the receiving electrode and a bypass electrode of the implantable device and to pass from the bypass electrode to the receiving electrode, for example, as described above with respect to FIGS. 11-13. In some aspects, the first output electrode and the second output electrode may be between the receiving electrode and the bypass electrode and the implantable device may stimulate an efferent nerve. In some aspects, the bypass electrode may be between the receiving electrode and the first output electrode, and the implantable device may stimulate an afferent nerve. In some aspects, the bypass electrode may be between the receiving electrode and the first output electrode, and the implantable device may stimulate a nerve containing both efferent and afferent fibers. The bypass channel may deliver alternating transcutaneous current to inhibit activation of the nerve. The bypass channel may deliver low frequency current for nerve stimulation, and the first channel may deliver rectified alternating current for nerve stimulation. The bypass channel may include a tuning resistor configured to determine the ratio of current applied by the bypass channel relative to the first channel.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

### LISTING OF EMBODIMENTS

Various exemplary embodiments of the present disclosure are further illustrated by the following listing of embodiments, which should not be construed to unduly limit the present disclosure:

1. An implantable device comprising: a receiving electrode; a first output electrode; a first channel, the implantable device being configured to receive a transcutaneous current having a first portion and a second portion, the first channel being configured to allow the first portion of the transcutaneous current to pass from the receiving electrode to the first output electrode and to inhibit the second portion of the transcutaneous current passing from the first output electrode to the receiving electrode; a second output electrode; and a second channel configured to allow the second portion of the transcutaneous current to pass from the second output electrode to the receiving electrode and to inhibit the first portion of the transcutaneous current passing from the receiving electrode to the second output electrode.

2. An implantable device according to embodiment 1, wherein the transcutaneous current is an alternating transcutaneous current, the first portion of the transcutaneous current is a positive portion of the alternating transcutaneous current, and the second portion of the transcutaneous current is a negative portion of the alternating transcutaneous current.

3. An implantable device according to any of embodiments 1 to 2, wherein the transcutaneous current comprises a series of bursts of alternating transcutaneous current, the first portion of the transcutaneous current is a positive portion of the series of bursts of alternating transcutaneous current, and the second portion of the transcutaneous current is a negative portion of the series of bursts of alternating transcutaneous current.

4. An implantable device according to any of embodiments 1 to 3, wherein the implantable device is configured to receive the transcutaneous current from an external generator.

5. An implantable device according to any of embodiments 1 to 4, wherein the transcutaneous current received by the implantable device is a fraction of a current applied by the external generator.

6. An implantable device according to any of embodiments 1 to 5, wherein the implantable device is configured to receive transcutaneous current from an external generator through electrically conductive coupling.

7. An implantable device according to any of embodiments 1 to 6, wherein the electrically conductive coupling is electrically coupling through conductive body tissue.

8. An implantable device according to any of embodiments 1 to 7, wherein the implantable device is configured to: receive a burst of alternating transcutaneous current from an external generator, the burst of alternating transcutaneous current burst having a positive portion and a negative portion; rectify the burst of alternating transcutaneous current to generate a positive current burst; rectify the burst of alternating transcutaneous current to generate a negative current burst; stimulate tissue at an implantation site of the first output electrode utilizing the positive current burst; and stimulate tissue at an implantation site of the second output electrode using the negative current burst.

9. An implantable device according to any of embodiments 1 to 8, wherein the implantable device is configured to: receive a high frequency transcutaneous current from an external generator, the high frequency transcutaneous current burst having a positive portion and a negative portion; rectify the high frequency transcutaneous current to generate a positive high frequency current; rectify the high frequency transcutaneous current to generate a negative high frequency current; stimulate tissue at an implantation site of the first output electrode utilizing the positive high frequency current; and stimulate tissue at an implantation site of the second output electrode using the negative high frequency current.

10. An implantable device according to any of embodiments 1 to 9, wherein the first channel comprises a first rectification element configured to inhibit current from flowing from the first output electrode to the receiving electrode.

11. An implantable device according to any of embodiments 1 to 10, wherein the second channel comprises a second rectification element configured to inhibit current from flowing from the receiving electrode to the second output electrode.

12. An implantable device according to any of embodiments 1 to 11, wherein the rectification element is a diode.

13. An implantable device according to any of embodiments 1 to 12, wherein the first channel comprises a capacitor coupled between the first output electrode and the receiving electrode, a diode coupled between the first output electrode and the receiving electrode, and a resistor coupled in parallel to the diode.

14. An implantable device according to any of embodiments 1 to 13, wherein the second channel comprises a second capacitor coupled between the second output electrode and the receiving electrode, a second diode coupled between the second output electrode and the receiving electrode, and a second resistor coupled in parallel to the second diode.

15. An implantable device according to any of embodiments 1 to 14, wherein the first output electrode and the second output electrode are configured to be implanted in proximity to a nerve of a subject, and wherein the receiving electrode is configured to be implanted in proximity to a surface of the skin of the subject.

16. An implantable device according to any of embodiments 1 to 15, wherein the implantable device is configured to apply monopolar stimulation to the nerve at an implantation site of the first output electrode, is configured to apply monopolar stimulation to the nerve at an implantation site of the second output electrode, and is configured to apply quasi-bipolar stimulation to the nerve between the implantation site of the first output electrode and the implantation site of the second output based on applying monopolar stimulation to the tissue at the first location and applying monopolar stimulation at the second location.

17. An implantable device according to any of embodiments 1 to 16, further comprising: a bypass electrode; and a bypass channel configured to allow transcutaneous current to pass between the receiving electrode and the bypass electrode, wherein the bypass channel is configured to allow current to pass from the receiving electrode to the bypass electrode and to pass from the bypass electrode to the receiving electrode.

18. An implantable device according to any of embodiments 1 to 17, wherein the first output electrode and the second output electrode are between the receiving electrode and the bypass electrode, and wherein the implantable device is configured to stimulate an efferent nerve.

19. An implantable device according to any of embodiments 1 to 18, wherein the bypass electrode is between the receiving electrode and the first output electrode, and wherein the implantable device is configured to stimulate an afferent nerve.

20. An implantable device according to any of embodiments 1 to 19, wherein the bypass electrode is between the receiving electrode and the first output electrode, and wherein the implantable device is configured to stimulate a nerve containing both efferent and afferent fibers.

21. An implantable device according to any of embodiments 1 to 20, wherein the bypass channel is configured to deliver alternating transcutaneous current to inhibit activation of the nerve.

22. An implantable device according to any of embodiments 1 to 21, wherein: the bypass channel is configured to deliver low frequency current for nerve stimulation, and the first channel is configured to deliver rectified alternating current for nerve stimulation.

23. An implantable device according to any of embodiments 1 to 22, wherein the bypass channel comprises a tuning resistor configured to determine the ratio of current applied by the bypass channel relative to the first channel.

24. An implantable device according to any of embodiments 1 to 23, further comprising an implantable lead, wherein: the implantable lead comprises a first end and a second end opposite the first end, the receiving electrode is located at or near the first end of the implantable lead, and the first output electrode or the second output electrode is located at or near the second end of the implantable lead.

25. An implantable device according to any of embodiments 1 to 24, wherein the first output electrode and the second output electrode are located at or near the second end of the implantable lead.

26. An implantable device according to any of embodiments 1 to 25, further comprising an implantable lead, wherein: the implantable lead comprises a first end, a first branch with a second end, and a second branch with a third end, the receiving electrode is located at or near the first end of the implantable lead, the first output electrode is located at or near the second end of the implantable lead, and the second output electrode is located at or near the third end of the implantable lead.

27. An implantable device according to any of embodiments 1 to 26, further comprising a receiver, wherein: the receiver comprises a cavity, the first channel comprises a rectification element configured to rectify current flowing in the first channel, and the rectification element is in the cavity of the receiver.

28. An implantable device according to any of embodiments 1 to 27, wherein the second channel comprises a second rectification element configured to rectify current flowing in the second channel, the second rectification element being in the cavity of the receiver.

29. An implantable device according to any of embodiments 1 to 28, wherein the receiver further comprises a lead connector.

30. An implantable device according to any of embodiments 1 to 29, further comprising: a receiver, the receiver comprising the receiving electrode and a cavity, and electrical elements configured to rectify the electrical current flowing in the first or in the second channel, the electrical elements being in the cavity of the receiver.

31. An implantable device according to any of embodiments 1 to 30, wherein the receiver comprises an electrical and mechanical connector configured to attach and electrically connect to a first end of an implanted lead.

32. An implantable device according to any of embodiments 1 to 31, wherein the first end of the lead electrically connects the first and the second channels to the second end of the lead.

33. An implantable device according to any of embodiments 1 to 32, wherein the transcutaneous current is a burst of alternating transcutaneous current with a carrier frequency between 5 kHz and 100 kHz.

34. An implantable device according to any of embodiments 1 to 33, wherein the transcutaneous current is a burst of alternating transcutaneous current with a carrier frequency between 100 kHz and 5 MHz.

35. An implantable device according to any of embodiments 1 to 34, wherein the transcutaneous current is a burst of alternating transcutaneous current with a carrier frequency between 1 kHz and 5 kHz.

36. An implantable device according to any of embodiments 1 to 35, wherein the transcutaneous current is a burst of alternating transcutaneous current and wherein the carrier frequency of the alternating transcutaneous current has a first value at a first time during the burst of alternating transcutaneous current and has a second value at a second time during the burst of alternating transcutaneous current, the first value being different than the second value.

37. An implantable device according to any of embodiments 1 to 36, wherein the transcutaneous current comprises a first burst of alternating transcutaneous current and a second burst of alternating transcutaneous current, wherein a carrier frequency of the transcutaneous current during the first burst is different than a carrier frequency of the transcutaneous current during the second burst.

38. An implantable device according to any of embodiments 1 to 37, wherein the transcutaneous current comprises a first burst of alternating transcutaneous current and a second burst of alternating transcutaneous current, wherein a carrier waveform of the transcutaneous current during the first burst is different than a carrier waveform of the transcutaneous current during the second burst.

39. A method of applying current to a tissue of a subject, the method comprising: receiving, by an implantable device, an alternating transcutaneous current; rectifying, by the implantable device, the alternating transcutaneous current to generate a positive current; rectifying, by the implantable device, the alternating transcutaneous current to generate a negative current; stimulating, by the implantable device, tissue at a first location utilizing the positive current; and stimulating, by the implantable device, tissue at a second location utilizing the negative current.

40. A method according to embodiment 39, wherein: the alternating transcutaneous current comprises a positive portion and a negative portion, the implantable device comprises a first channel configured to allow the positive portion of the alternating transcutaneous current to pass from a receiving electrode of the implantable device to a first output electrode of the implantable device and to inhibit the negative portion of the alternating transcutaneous current passing from the first output electrode to the receiving electrode, the first output electrode being at the first location, the implantable device comprises a second channel configured to allow the negative portion of the transcutaneous current to pass from a second output electrode of the implantable device to the receiving electrode and to inhibit the positive portion of the transcutaneous current passing from the receiving electrode to the second output electrode.

41. A method according to any of embodiments 39 to 40, wherein the first output electrode and the second output electrode are configured to be implanted in proximity to a nerve of a subject, and wherein the receiving electrode is configured to be implanted in proximity to a surface of the skin of the subject.

42. A method according to any of embodiments 39 to 41, further comprising: applying, by the implantable device, an unrectified current to tissue at a third location.

43. A method according to any of embodiments 39 to 42, wherein the implantable device comprises a bypass channel configured to allow transcutaneous current to pass between a receiving electrode of the implantable device and a bypass electrode of the implantable device and to pass from the bypass electrode to the receiving electrode.

44. A method according to any of embodiments 39 to 43, wherein the first output electrode and the second output electrode are between the receiving electrode and the bypass electrode, and wherein the implantable device is configured to stimulate an efferent nerve.

45. A method according to any of embodiments 39 to 44, wherein the bypass electrode is between the receiving electrode and the first output electrode, and wherein the implantable device is configured to stimulate an afferent nerve.

46. A method according to any of embodiments 39 to 45, wherein the bypass electrode is between the receiving electrode and the first output electrode, and wherein the implantable device is configured to stimulate a nerve containing both efferent and afferent fibers.

47. A method according to any of embodiments 39 to 46, wherein the bypass channel is configured to deliver alternating transcutaneous current to inhibit activation of the nerve.

48. A method according to any of embodiments 39 to 47, wherein: the bypass channel is configured to deliver low frequency current for nerve stimulation, and the first channel is configured to deliver rectified alternating current for nerve stimulation.

49. A method according to any of embodiments 39 to 48, wherein the bypass channel comprises a tuning resistor configured to determine the ratio of current applied by the bypass channel relative to the first channel.

50. A method according to any of embodiments 39 to 49, wherein the alternating transcutaneous current comprises a series of bursts of alternating transcutaneous current, the positive portion of the alternating transcutaneous current is a positive portion of the series of bursts of alternating transcutaneous current, and the negative portion of the transcutaneous current is a negative portion of the series of bursts of alternating transcutaneous current.

51. A method according to any of embodiments 39 to 50, wherein the alternating transcutaneous current is received from an external generator.

52. A method according to any of embodiments 39 to 51, wherein the alternating transcutaneous current received by the implantable device is a fraction of a current applied by the external generator.

53. A method according to any of embodiments 39 to 52, wherein the alternating transcutaneous current is received through electrically conductive coupling.

54. A method according to any of embodiments 39 to 53, wherein the electrically conductive coupling is electrically coupling through conductive body tissue.

55. A method according to any of embodiments 39 to 54, wherein the alternating transcutaneous current is high frequency alternating transcutaneous current, the positive current is a positive high frequency current, and the negative current is a negative high frequency current.

56. A method according to any of embodiments 39 to 55, wherein stimulating tissue at the first location comprises applying monopolar stimulation to the tissue at the first location, and wherein stimulating tissue at the second location comprises applying monopolar stimulation to the tissue at the second location, the method further comprising: applying quasi-bipolar stimulation between the first location and the second location based on applying monopolar stimulation to the tissue at the first location and applying monopolar stimulation at the second location.

57. A method according to any of embodiments 39 to 56, wherein the alternating transcutaneous current is a burst of alternating transcutaneous current with a carrier frequency between 5 kHz and 100 kHz.

58. A method according to any of embodiments 39 to 57, wherein the alternating transcutaneous current is a burst of alternating transcutaneous current with a carrier frequency between 100 kHz and 5 MHz.

59. A method according to any of embodiments 39 to 58, wherein the alternating transcutaneous current is a burst of alternating transcutaneous current with a carrier frequency between 1 kHz and 5 kHz.

60. A method according to any of embodiments 39 to 59, wherein the alternating transcutaneous current is a burst of alternating transcutaneous current and wherein the carrier frequency of the alternating transcutaneous current has a first value at a first time during the burst of alternating transcutaneous current and has a second value at a second time during the burst of alternating transcutaneous current, the first value being different than the second value.

61. A method according to any of embodiments 39 to 60, wherein the transcutaneous current comprises a first burst of alternating transcutaneous current and a second burst of alternating transcutaneous current, wherein a carrier frequency of the transcutaneous current during the first burst is different than a carrier frequency of the transcutaneous current during the second burst.

62. A method according to any of embodiments 39 to 61, wherein the transcutaneous current comprises a first burst of alternating transcutaneous current and a second burst of alternating transcutaneous current, wherein a carrier waveform of the transcutaneous current during the first burst is different than a carrier waveform of the transcutaneous current during the second burst.

What is claimed is:

1. An implantable device comprising:
a receiving electrode;
a first output electrode;
a first channel, the implantable device being configured to receive a transcutaneous current having a first portion and a second portion, the first channel being configured to allow the first portion of the transcutaneous current to pass from the receiving electrode to the first output electrode and to inhibit the second portion of the transcutaneous current passing from the first output electrode to the receiving electrode;
a second output electrode; and
a second channel configured to allow the second portion of the transcutaneous current to pass from the second output electrode to the receiving electrode and to inhibit the first portion of the transcutaneous current passing from the receiving electrode to the second output electrode.

2. The implantable device of claim 1, wherein the transcutaneous current is an alternating transcutaneous current, the first portion of the transcutaneous current is a positive portion of the alternating transcutaneous current, and the second portion of the transcutaneous current is a negative portion of the alternating transcutaneous current.

3. The implantable device of claim 1, wherein the transcutaneous current comprises a series of bursts of alternating transcutaneous current, the first portion of the transcutaneous current is a positive portion of the series of bursts of alternating transcutaneous current, and the second portion of the transcutaneous current is a negative portion of the series of bursts of alternating transcutaneous current.

4. The implantable device of claim 1, wherein the implantable device is configured to receive the transcutaneous current from an external generator.

5. The implantable device of claim 4, wherein the transcutaneous current received by the implantable device is a fraction of a current applied by the external generator.

6. The implantable device of claim 1, wherein the implantable device is configured to receive transcutaneous current from an external generator through electrically conductive coupling.

7. The implantable device of claim 6 wherein the electrically conductive coupling is electrically coupling through conductive body tissue.

8. The implantable device of claim 1, wherein the implantable device is configured to:
receive a burst of alternating transcutaneous current from an external generator, the burst of alternating transcutaneous current burst having a positive portion and a negative portion;
rectify the burst of alternating transcutaneous current to generate a positive current burst;
rectify the burst of alternating transcutaneous current to generate a negative current burst;
stimulate tissue at an implantation site of the first output electrode utilizing the positive current burst; and
stimulate tissue at an implantation site of the second output electrode using the negative current burst.

9. The implantable device of claim 1, wherein the implantable device is configured to:
receive a high frequency transcutaneous current from an external generator, the high frequency transcutaneous current burst having a positive portion and a negative portion;
rectify the high frequency transcutaneous current to generate a positive high frequency current;
rectify the high frequency transcutaneous current to generate a negative high frequency current;
stimulate tissue at an implantation site of the first output electrode utilizing the positive high frequency current; and
stimulate tissue at an implantation site of the second output electrode using the negative high frequency current.

10. The implantable device of claim 1, wherein the first channel comprises a first rectification element configured to inhibit current from flowing from the first output electrode to the receiving electrode.

11. The implantable device of claim 10, wherein the second channel comprises a second rectification element configured to inhibit current from flowing from the receiving electrode to the second output electrode.

12. The implantable device of claim 10, wherein the rectification element is a diode.

13. The implantable device of claim 1, wherein the first channel comprises a capacitor coupled between the first output electrode and the receiving electrode, a diode coupled between the first output electrode and the receiving electrode, and a resistor coupled in parallel to the diode.

14. The implantable device of claim 13, wherein the second channel comprises a second capacitor coupled between the second output electrode and the receiving electrode, a second diode coupled between the second output electrode and the receiving electrode, and a second resistor coupled in parallel to the second diode.

15. The implantable device of claim 1, wherein the first output electrode and the second output electrode are configured to be implanted in proximity to a nerve of a subject, and wherein the receiving electrode is configured to be implanted in proximity to a surface of the skin of the subject.

16. The implantable device of claim 1, wherein the implantable device is configured to apply monopolar stimulation to the nerve at an implantation site of the first output electrode, is configured to apply monopolar stimulation to the nerve at an implantation site of the second output electrode, and is configured to apply quasi-bipolar stimulation to the nerve between the implantation site of the first output electrode and the implantation site of the second output based on applying monopolar stimulation to the tissue at the first location and applying monopolar stimulation at the second location.

17. The implantable device of claim 1, further comprising:
a bypass electrode; and
a bypass channel configured to allow transcutaneous current to pass between the receiving electrode and the bypass electrode, wherein the bypass channel is configured to allow current to pass from the receiving electrode to the bypass electrode and to pass from the bypass electrode to the receiving electrode.

18. The implantable device of claim 17, wherein the first output electrode and the second output electrode are between the receiving electrode and the bypass electrode, and wherein the implantable device is configured to stimulate an efferent nerve.

19. The implantable device of claim 17, wherein the bypass electrode is between the receiving electrode and the first output electrode, and wherein the implantable device is configured to stimulate an afferent nerve.

20. The implantable device of claim 17, wherein the bypass electrode is between the receiving electrode and the first output electrode, and wherein the implantable device is configured to stimulate a nerve containing both efferent and afferent fibers.

21. The implantable device of claim 17, wherein the bypass channel is configured to deliver alternating transcutaneous current to inhibit activation of the nerve.

22. The implantable device of claim 17, wherein:
the bypass channel is configured to deliver low frequency current for nerve stimulation, and
the first channel is configured to deliver rectified alternating current for nerve stimulation.

23. The implantable device of claim 17, wherein the bypass channel comprises a tuning resistor configured to determine the ratio of current applied by the bypass channel relative to the first channel.

24. The implantable device of claim 1, further comprising an implantable lead, wherein:
the implantable lead comprises a first end and a second end opposite the first end,
the receiving electrode is located at or near the first end of the implantable lead, and
the first output electrode or the second output electrode is located at or near the second end of the implantable lead.

25. The implantable device of claim 24, wherein the first output electrode and the second output electrode are located at or near the second end of the implantable lead.

26. The implantable device of claim 1, further comprising an implantable lead, wherein:
the implantable lead comprises a first end, a first branch with a second end, and a second branch with a third end,
the receiving electrode is located at or near the first end of the implantable lead,
the first output electrode is located at or near the second end of the implantable lead, and
the second output electrode is located at or near the third end of the implantable lead.

27. The implantable device of claim 1, further comprising a receiver, wherein:
the receiver comprises a cavity,
the first channel comprises a rectification element configured to rectify current flowing in the first channel, and
the rectification element is in the cavity of the receiver.

28. The implantable device of claim 27, wherein the second channel comprises a second rectification element configured to rectify current flowing in the second channel, the second rectification element being in the cavity of the receiver.

29. The implantable device of claim 27, wherein the receiver further comprises a lead connector.

30. The implantable device of claim 1, further comprising:
a receiver, the receiver comprising the receiving electrode and a cavity, and
electrical elements configured to rectify the electrical current flowing in the first or in the second channel, the electrical elements being in the cavity of the receiver.

31. The implantable device of claim 30, wherein the receiver comprises an electrical and mechanical connector configured to attach and electrically connect to a first end of an implanted lead.

32. The implantable device of claim 31, wherein the first end of the lead electrically connects the first and the second channels to the second end of the lead.

33. The implantable device of claim 1, wherein the transcutaneous current is a burst of alternating transcutaneous current with a carrier frequency between 5 kHz and 100 kHz.

34. The implantable device of claim 1, wherein the transcutaneous current is a burst of alternating transcutaneous current with a carrier frequency between 100 kHz and 5 MHz.

35. The implantable device of claim 1, wherein the transcutaneous current is a burst of alternating transcutaneous current with a carrier frequency between 1 kHz and 5 kHz.

36. The implantable device of claim 1, wherein the transcutaneous current is a burst of alternating transcutaneous current and wherein the carrier frequency of the alternating transcutaneous current has a first value at a first time during the burst of alternating transcutaneous current and has a second value at a second time during the burst of alternating transcutaneous current, the first value being different than the second value.

37. The implantable device of claim 1, wherein the transcutaneous current comprises a first burst of alternating transcutaneous current and a second burst of alternating transcutaneous current, wherein a carrier frequency of the transcutaneous current during the first burst is different than a carrier frequency of the transcutaneous current during the second burst.

38. The implantable device of claim 1, wherein the transcutaneous current comprises a first burst of alternating transcutaneous current and a second burst of alternating transcutaneous current, wherein a carrier waveform of the transcutaneous current during the first burst is different than a carrier waveform of the transcutaneous current during the second burst.

39. A method of applying current to a tissue of a subject, the method comprising:
receiving, by an implantable device, an alternating transcutaneous current;
rectifying, by the implantable device, the alternating transcutaneous current to generate a positive current;
rectifying, by the implantable device, the alternating transcutaneous current to generate a negative current;
stimulating, by the implantable device, tissue at a first location utilizing the positive current; and
stimulating, by the implantable device, tissue at a second location utilizing the negative current.

40. The method of claim 39, wherein:
the alternating transcutaneous current comprises a positive portion and a negative portion,
the implantable device comprises a first channel configured to allow the positive portion of the alternating transcutaneous current to pass from a receiving electrode of the implantable device to a first output electrode of the implantable device and to inhibit the negative portion of the alternating transcutaneous current passing from the first output electrode to the receiving electrode, the first output electrode being at the first location,
the implantable device comprises a second channel configured to allow the negative portion of the transcutaneous current to pass from a second output electrode of the implantable device to the receiving electrode and to inhibit the positive portion of the transcutaneous current passing from the receiving electrode to the second output electrode.

41. The method of claim 40, wherein the first output electrode and the second output electrode are configured to be implanted in proximity to a nerve of a subject, and wherein the receiving electrode is configured to be implanted in proximity to a surface of the skin of the subject.

42. The method of claim 40, further comprising:
applying, by the implantable device, an unrectified current to tissue at a third location.

43. The method of claim 42, wherein the implantable device comprises a bypass channel configured to allow transcutaneous current to pass between a receiving electrode of the implantable device and a bypass electrode of the implantable device and to pass from the bypass electrode to the receiving electrode.

44. The method of claim 43, wherein the first output electrode and the second output electrode are between the receiving electrode and the bypass electrode, and wherein the implantable device is configured to stimulate an efferent nerve.

45. The method of claim 43, wherein the bypass electrode is between the receiving electrode and the first output electrode, and wherein the implantable device is configured to stimulate an afferent nerve.

46. The method of claim 43, wherein the bypass electrode is between the receiving electrode and the first output electrode, and wherein the implantable device is configured to stimulate a nerve containing both efferent and afferent fibers.

47. The method of claim 43, wherein the bypass channel is configured to deliver alternating transcutaneous current to inhibit activation of the nerve.

48. The method of claim 43, wherein:
the bypass channel is configured to deliver low frequency current for nerve stimulation, and
the first channel is configured to deliver rectified alternating current for nerve stimulation.

49. The method of claim 43, wherein the bypass channel comprises a tuning resistor configured to determine the ratio of current applied by the bypass channel relative to the first channel.

50. The method of claim 39, 1 wherein the alternating transcutaneous current comprises a series of bursts of alternating transcutaneous current, the positive portion of the alternating transcutaneous current is a positive portion of the series of bursts of alternating transcutaneous current, and the negative portion of the transcutaneous current is a negative portion of the series of bursts of alternating transcutaneous current.

51. The method of claim 39, wherein the alternating transcutaneous current is received from an external generator.

52. The method of claim 51, wherein the alternating transcutaneous current received by the implantable device is a fraction of a current applied by the external generator.

53. The method of claim 39, wherein the alternating transcutaneous current is received through electrically conductive coupling.

54. The method of claim 53, wherein the electrically conductive coupling is electrically coupling through conductive body tissue.

55. The method of claim 39, wherein the alternating transcutaneous current is high frequency alternating transcutaneous current, the positive current is a positive high frequency current, and the negative current is a negative high frequency current.

56. The method of claim 39, wherein stimulating tissue at the first location comprises applying monopolar stimulation to the tissue at the first location, and wherein stimulating tissue at the second location comprises applying monopolar stimulation to the tissue at the second location, the method further comprising:
applying quasi-bipolar stimulation between the first location and the second location based on applying monopolar stimulation to the tissue at the first location and applying monopolar stimulation at the second location.

57. The method of claim 39, wherein the alternating transcutaneous current is a burst of alternating transcutaneous current with a carrier frequency between 5 kHz and 100 kHz.

58. The method of claim 39, wherein the alternating transcutaneous current is a burst of alternating transcutaneous current with a carrier frequency between 100 kHz and 5 MHz.

59. The method of claim 39, 1 wherein the alternating transcutaneous current is a burst of alternating transcutaneous current with a carrier frequency between 1 kHz and 5 kHz.

60. The method of claim 39, wherein the alternating transcutaneous current is a burst of alternating transcutaneous current and wherein the carrier frequency of the alternating transcutaneous current has a first value at a first time during the burst of alternating transcutaneous current and has a second value at a second time during the burst of alternating transcutaneous current, the first value being different than the second value.

61. The method of claim 39, wherein the transcutaneous current comprises a first burst of alternating transcutaneous current and a second burst of alternating transcutaneous current, wherein a carrier frequency of the transcutaneous current during the first burst is different than a carrier frequency of the transcutaneous current during the second burst.

62. The method of claim 39, wherein the transcutaneous current comprises a first burst of alternating transcutaneous current and a second burst of alternating transcutaneous current, wherein a carrier waveform of the transcutaneous current during the first burst is different than a carrier waveform of the transcutaneous current during the second burst.
